(19) United States
(12) Patent Application Publication
(10) Pub. No.: US 2018/0346577 A1
(43) Pub. Date: Dec. 6, 2018

(54) TREATMENT OF NEUROINFLAMMATORY DISEASE

(71) Applicant: The Board of Trustees of the Leland Stanford Junior University, Stanford, CA (US)

(72) Inventors: Bahareh Ajami, Mountain View, CA (US); Lawrence Steinman, Stanford, CA (US)

(21) Appl. No.: 15/993,172

(22) Filed: May 30, 2018

Related U.S. Application Data
(60) Provisional application No. 62/512,457, filed on May 30, 2017.

(51) Int. Cl. C07K 16/28; A61K 39/395; A61K 38/21; A61P 25/02

(52) U.S. Cl. CPC C07K 16/2839; A61K 39/39541; A61K 2039/505; A61P 25/02; C07K 2317/76; A61K 38/215

(57) ABSTRACT

Provided herein are methods and compositions for treating inflammatory diseases by administering to the subject an effective dose on an anti-α₅ agent.

### CROSS REFERENCE

This application claims benefit of U.S. Provisional Patent Application No. 62/512,457, filed May 30, 2017, which application is incorporated herein by reference in its entirety.

### BACKGROUND

Multiple sclerosis (MS) is the most prevalent inflammatory disease of the brain and spinal cord in Europe and North America. More than one million are affected worldwide, including 400,000 in the US. Symptoms often commence in young adulthood and include motor paralysis, visual disturbances and blindness, bowel and bladder incontinence, sensory loss, and incoordination and ataxia. The first line of approved therapies in the US are glatiramer acetate (Copaxone), IFN-β1a (Avonex and Rebif), and IFN-β1b (Betaseron and Extavia) and the second line of approved therapies are mitoxantrone (Novantrone) and natalizumab (Tysabri). Recently, fingolomid, terflunimide, and dimethyl fumarate, have been separately approved by the US FDA as new options of orally administered first line of therapy for the treatment of relapsing MS.

Current approved treatments for MS are limited in their efficacy, and are costly. Therefore, there is still an urgent need to find better effective treatment for MS. Natalizumab, a humanized antibody to alpha4 integrin, is the most potent treatment but is burdened with serious life threatening side effect. More than 1 in 500 individuals treated with natalizumab have developed a devastating opportunistic infection of the brain, progressive multifocal leukoencephalopathy (PML). This adverse effect is due to ability of this drug to block the homing of T lymphocytes as well as monocytes to the CNS. However, the T cells are required to fight the reactivation of John Cunningham (JC) virus infections. T cell immunity to JC prevents the appearance of PML that results from JC viral infection.

Improved methods of treatment that reduce these undesirable side effects are provided herein.

### SUMMARY

Therapeutic methods are provided for the treatment of inflammatory diseases, including neuroinflammatory disease such as, for example, neuroinflammatory demyelinating autoimmune diseases, such as multiple sclerosis (MS) and neuromyelitis optica (NMO), etc., and also including treatment of amyotrophic lateral sclerosis (ALS). In the methods of the invention, an effective dose of one or a cocktail of antagonist(s) to α5 integrin (CD49e) is administered to a subject suffering from a neurological inflammatory diseases, in a dose effective to stabilize or reduce clinical symptoms of the disease. As shown herein, specific myeloid cell populations associated with central nervous system (CNS) disease express CD49e during disease states and development of disease states. An overview of the cell populations is provided in Table 4. Populations A, B, and C correspond to microglial cells, which upregulate CD49e in ALS disease. Populations D, E, F, G and H are infiltrating monocytes, which are associated with neuroinflammatory disease, and which express CD49e during specific stages in the development of neuroinflammatory demyelinating such as MS, EAE, etc.

In various aspects and embodiments, the methods may include administering to a subject suffering from a neurological inflammatory diseases an effective dose of an antibody that specifically binds to CD49e, where the treatment reduces or stabilizes clinical symptoms of the disease. In some embodiments the anti-CD49e agent is combined with a second therapeutic agent, including without limitation a statin, cytokine, antibody, copaxone, fingolomid, etc. In some embodiments the anti-CD49e agent is combined with a statin in a dose effective to control serum cholesterol levels.

In one embodiment, provided is a package (for example a box, a bottle or a bottle and box) that includes an anti-CD49e agent and a package insert or label that indicates that the anti-α₅ agent is to be administered to a patient for the treatment of a neurological inflammatory disease, e.g. MS, NMO, ALS, etc.

In one embodiment, provided is a method of treating a neurological inflammatory disease, e.g. MS, NMO, etc. or ALS that includes administering to a patient an effective dose of an anti-α₅ agent alone or in combination with a statin, or in combination with one or more therapeutic compounds, including without limitation a cytokine; an antibody, e.g. tysabri; fingolimod (Gilenya); copaxone, etc. The effective dose of each drug in a combination therapy may be lower than the effective dose of the same drug in a monotherapy. In some embodiments the combined therapies are administered concurrently. In some embodiments the two therapies are phased, for example where one compound is initially provided as a single agent, e.g. as maintenance, and where the second compound is administered during a relapse, for example at or following the initiation of a relapse, at the peak of relapse, etc.

In an embodiment, provided is a method for treating amyotrophic lateral sclerosis, which is shown herein to have a high content of CD49e⁺ myeloid cells in the spinal cord. An effective dose of one or a cocktail of antagonist(s) to CD49e is administered to stabilize or reduce clinical symptoms of ALS. In some embodiments the antagonist(s) to CD49e are delivered to cerebrospinal fluid, e.g. by intrathecal delivery, etc. In some embodiments the delivery is systemic.

In another embodiment, provided is a method for removing tattoos, by administering one or a cocktail of antagonist(s) to CD49e to an individual for removal of a tattoo that is desired to be removed, where the antagonist to CD49e reduces activity of macrophages that contribute to the permanence of a tattoo. In some embodiments the antagonist to CD49e is delivered locally to the site of a tattoo. In some embodiments the antagonist(s) to CD49e is delivered by a sustained release formulation to the site of the tattoo. In other embodiments the delivery is systemic.

Alternatively the anti-CD49e agent is initially provided as a single agent, e.g. as maintenance, and the additional agent is administered during a relapse, for example at or following the initiation of a relapse, at the peak of relapse, etc. In certain of such embodiments, a package is provided comprising includes an anti-CD49e agent, and one or more second therapeutic compounds, and a package insert or label that indicates that the anti-CD49e agent is to be administered in combination with the second compound to a patient for the treatment of a neurological inflammatory disease.

In some embodiments of the invention, the patient is analyzed for responsiveness to therapy, where the selection of therapeutic agents is based on such analysis. The efficacy of immunomodulatory treatments on neurological inflammatory disease of the central nervous system, e.g. multiple sclerosis, neuromyelitis optica, EAE, etc., depends on whether a patient has a predominantly TH1-type disease subtype, or a predominantly TH17-type disease subtype. Patients can be classified into subtypes by determining the levels of markers, including IL-17; endogenous β-interferon, IL-23, PDGFBB, sFAS ligand, M-CSF, MIP1α, TNF-β, IFNα, IL-1RA, MCP-1, IL-2, IL-6, IL-8, FGFβ, IL-7, TGF-β, IFNβ, IL-13, IL-17F, EOTAXIN, IL-1a, MCP-3, LIF, NGF, RANTES, IL-5, MIP1b, IL-12p70, and HGF, etc. Cytokines such as β-interferon may be administered to individuals having a predominantly TH1-type disease subtype in combination with an anti-CD49e agent.

In some embodiments, where the condition to be treated is a neuroinflammatory condition, e.g. MS, EAE, NMO, etc., a patient may be treated when CD49e monocyte populations infiltrate the CNS. A summary of the changes in populations that correspond to stages of disease is shown in FIG. 5C. For example, an increase may be observed where the frequency is greater than about 1%, greater than about 2%, greater than about 3% of the total cells present in CSF. An increase can also be measured relative to a normal control, or to a reference value corresponding to the levels in a normal control. The number of cells in a population producing two or more cytokines, e.g. expressing two or more of TNFα, GM-CSF, IL-6, IL-10 and TGFβ, as shown in FIG. 12, is also increased in disease relative to healthy controls. In some embodiments the cells present in the CSF are measured from a sample from a patient for markers indicative of infiltrating myeloid cells, and the presence of changes, particularly changes in cells expressing CD49e, utilized as the basis for treatment.

The presence of increased numbers of cells in populations D, G and H in the CNS is indicative of pre-symptomatic disease. This increase provides a useful biomarker for pre-symptomatic disease, and a patient may be treated with an anti-CD49e agent when an increase is observed. The presence of increased numbers of cells in populations D, E, F and G is pronounced in the CNS at the onset of disease, and a patient may be treated with an anti-CD49e agent when such an increase is observed. At peak of disease an increase in population D is particularly pronounced, although the other populations are also increased, and a patient may be treated with an anti-CD49e agent when such an increase is observed. Interestingly, recovery is associated with increased number of population F cells expressing single or no cytokines TNFα, IL-6, TGFβ.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Schematic representation of the experimental strategy. Immune response profiles were analyzed in Healthy, five different clinical stages of experimental autoimmune encephalomyelitis (EAE) and R6/2 transgenic mice a well-established Huntington's disease (HD) mouse model. Single-cell suspensions from CNS (brain and spinal cord) and whole blood of each condition were prepared as described in Material and Methods. Individual samples were simultaneously processed by using the barcoding strategy (Material and Methods). Barcoded samples were pooled, stained with a panel of 39 antibodies FIGS. 12, 2 and 3 and Material and Methods), and analyzed by mass cytometry (CyTOF). Raw mass cytometry data were normalized for signal variation over time and debarcoded and analyzed using the X-shift algorithm, a nonparametric clustering method that automatically identifies cell populations by searching for local maxima of cell event density in the multidimensional marker space. The result is displayed as a minimum-spanning tree (MST) layout. Each experiment performed seven to ten times independently. In each experiment, tissues from ten mice were pooled in order to provide enough cell number.

FIG. 2A-2D. Data-driven, unsupervised clustering defines three distinct myeloid populations in CNS. FIG. 2A Composite CNS Minimum Spanning Tree (MST) of X-shift clusters constructed by combining CNS samples from all the conditions and their biological replicates in comparison to composite MST from blood samples demonstrates three myeloid (CD11b+) populations that are unique to CNS (Population A, B and C). FIG. 2B Manual gating based on markers defined by the X-shift/DMT algorithm confirmed the existence of populations A, B and C. FIG. 2C-2D MSTs FIG. 2C, illustrating X-shift clustering frequencies of each condition, and the bar graph FIG. 2D presenting average frequency analysis based on manual gating, demonstrate that populations A, B and C are present in both EAE and HD models in CNS. Error bars represent standard deviation across replicates. Color coded scale represents the arsinh (x/5) transformed CyTOF signal intensity of each marker as described in Material and Methods. Data are from five or six independent experiments.

FIG. 3A-3D. Dynamic of key signaling molecules of immune activation pathways in CNS-residents myeloid cells. Line graphs show median of average expression level of raw CyTOF signal intensity per population. The error bars represent standard error (SE) across biological replicates (data from five or six independent experiments). The grey area represents the interquartile range of the given signaling molecule in all cells in a sample, averaged across replicates, and thus indicates the overall expression range for each marker.

FIG. 4A-4D. Single-cell analysis of cytokine production by three CNS-resident myeloid subsets in response to different disease conditions. FIG. 4A Distribution plots (Violin plots) shows the expression levels of indicated intracellular cytokines grouped by disease condition and cellular population. Plots were created in Mathematica. Plots show arsinh(x/5) transformed CyTOF signal intensity. FIG. 4B-4D Analysis of cytokine co-expression in CNS-resident myeloid cells in healthy and diseased states demonstrating heterogeneous subsets in each subpopulation. Percentages of single-cells expressing zero, one or two cytokines are represented in a stacked bar graph. Data are from three independent experiments.

FIG. 5A-5D. Kinetics of Blood-Derived Monocyte Migration to CNS in Inflammatory versus Degenerative conditions. FIG. 5A Composite MST reveals five distinct Ly6C⁺Ly6G⁻ myeloid populations (blood-derived monocytes) in CNS. FIG. 5B Each population is confirmed by manual gating based on markers defined by the X-shift/DMT algorithm. FIG. 5C Average frequency analysis based on manual gating demonstrates that there is a minimum accumulation of blood-derived monocytes in healthy and neurodegenerative conditions. In EAE disease, different blood-derived monocytes subsets accumulated depending on the disease state. Error bars represent standard deviation across replicates. FIG. 5D Blood-derived monocytes express MHC-11. Data are from five or six independent experiments.

FIG. 6A-6C. Differential Expression of Cell Surface Phenotype and Signaling molecules On Infiltrating versus Resident Myeloid Cells in inflammatory condition. FIG. 6A Cell Surface Phenotype analysis reveals high expression of CD49d (4 integrin) and CD49e (5 integrin) only on infiltrating monocytes compared to CNS-resident myeloid cells. CD49e is only expressed on monocyte whereas CD49d is also expressed on T cells and DCs. FIG. 6B Average clinical score for EAE mice treated with an antibody against CD49e (α5 integrin) compared to an isotype control. Mice (n=5) treated with an antibody against CD49e (α5 integrin) compared to an isotype control exhibit a delay in development of the disease onset and significantly reduced overall disease severity in treated animals. The experiment was concluded due to high morbidity of control mice. The error bars represent standard error (SE). FIG. 6C Heat map representing the comparison of median of average expression level of raw CyTOF signal intensity for each signaling molecule between CNS-resident myeloid cells and blood-derived monocytes in presymptomatic, onset and peak when all five monocyte subsets are present. The color representing the signaling molecule expression ranges from blue (undetectable) to white (intermediate) to red (maximum). Mass cytometry data are from five or six independent experiments.

FIG. 7A-7B. Single-cell analysis of cytokine production by different blood-derived monocyte subsets in response to different disease conditions. FIG. 7A Distribution plots of the levels of indicated intracellular cytokines grouped by disease condition and cellular population. Plots were created in Mathematica. Values are scaled by arsinh [x/5]. FIG. 7B X-shift analysis of the co-expression of cytokines in blood-derived monocyte subsets suggests that each subpopulation contains heterogeneous subsets depending on each disease conditions. Percentages of single-cells expressing zero, one, two, three or four cytokines are represented in a stacked bar graph. Data are from three independent experiments.

FIG. 8. Similarity in expression of several markers in three CNS-resident myeloid subsets. Populations A, B and C expressed different levels of CD88, MHC class I (H2), TAM receptor tyrosine kinases Mer (MerTK), and the newly introduced microglia markers 4D4 and fcrls.

FIG. 9. Variation in expression of several markers in three CNS-resident myeloid subsets. Differential expression of a number of markers were detected in three CNS-resident myeloid cells. Populations B and C expressed different levels of CD80, TAM receptor Axl, T-cell immunoglobulin mucin protein 4 (TIM4), CD274 (PD-L1), CD195 (CCR5), CD194 (CCR4), and low levels of CD206 and TREM2. Population A lacked the expression of all these markers.

FIG. 10. Expression of YFP in CNS-resident myeloid subsets. In Healthy conditional Cx3cr^{creER} Rosa26-YFP mice, populations A and B (the only two populations that exist in healthy condition) were manually gated and the expression of YFP was confirmed in them. The gating strategy is described in FIG. 2b.

FIG. 11. Variation in expression of several markers in five blood-derived monocyte subsets. Differential expression of a number of markers were detected in blood-derived monocyte subsets. Populations D and E compared to the other three subsets have a higher expression of phagocytic receptors like the TAM receptor tyrosine kinases Mer, Axl, costimulatory molecules (CD80, CD86), receptors involved in purinergic signaling (CD38, CD39), and TREM2 as well as CD206.

FIG. 12. Expression of cytokines in myeloid populations D-H during neuroinflammatory disease.

FIG. 13. CD49e expression is increased in microglia populations at disease end-stage in mice over-expressing human mutant superoxide dismutase 1 (mSOD), a murine model of ALS.

FIG. 14. Frequency of microglial cell populations in CSF during development of mSOD1 disease.

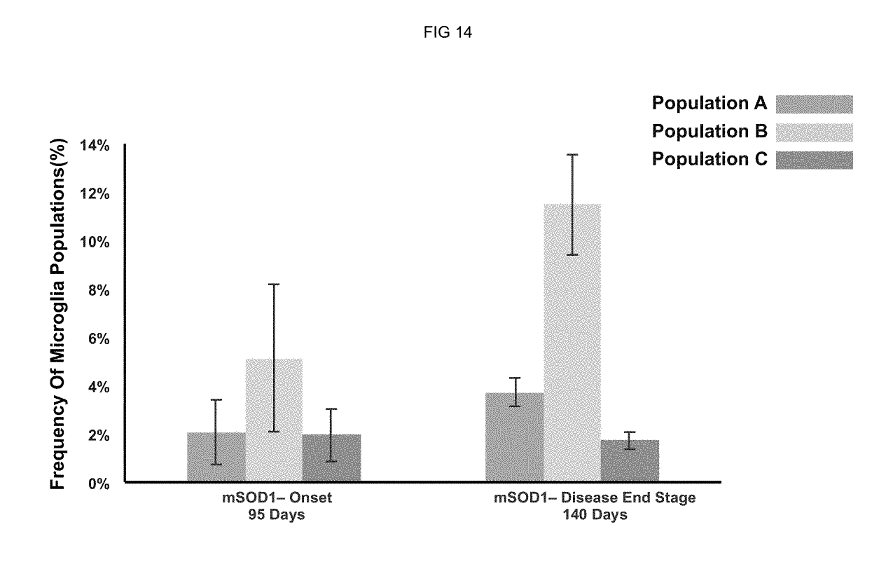

FIG. 15. Expression of cytokines in microglial cells during development of mSOD1 disease.

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present inventions have been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

Improvement in the use of disease-modifying therapies in neurological diseases is of great clinical interest. In certain aspects and embodiments the present methods and compositions address this need.

The subject methods may be used for prophylactic or therapeutic purposes. As used herein, the term “treating” is used to refer to both prevention of relapses, and treatment of pre-existing conditions. For example, the prevention of autoimmune disease may be accomplished by administration of the agent prior to development of a relapse. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. The treatment of ongoing disease, where the treatment stabilizes or improves the clinical symptoms of the patient, is of particular interest.

“Inhibiting” the onset of a disorder shall mean either lessening the likelihood of the disorders onset, or preventing the onset of the disorder entirely. Reducing the severity of a relapse shall mean that the clinical indicia associated with a relapse are less severe in the presence of the therapy than in an untreated disease. As used herein, onset may refer to a relapse in a patient that has ongoing relapsing remitting disease. The methods of the invention are specifically applied to patients that have been diagnosed with neurological inflammatory disease. Treatment is aimed at the treatment or reducing severity of relapses, which are an exacerbation of a pre-existing condition.

“Diagnosis” as used herein generally includes determination of a subject's susceptibility to a disease or disorder, determination as to whether a subject is presently affected by a disease or disorder, prognosis of a subject affected by a disease or disorder (e.g., identification of disease states, stages of MS, or responsiveness of MS to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “individual,” “subject,” “host,” and “patient,” used interchangeably herein and refer to any mammalian subject for whom diagnosis, treatment, or therapy is desired, for example humans, non-human primate, mouse, rat, guinea pig, rabbit, etc.

“Inhibiting” the expression of a gene in a cell shall mean either lessening the degree to which the gene is expressed, or preventing such expression entirely.

Integrins are heterodimeric transmembrane receptors that mediate cell-adhesion. Most integrins bind extracellular matrix (ECM) glycoproteins such as laminins and collagens in basement membranes or connective tissue components like fibronectin. Many of the ECM proteins that bind to integrins share a common integrin-binding motif, Arg-Gly-Asp (RGD), which is present in fibronectin, vitronectin, fibrinogen, and many others. Others bind counterreceptors on neighboring cells, bacterial polysaccharides, or viral coat proteins. Integrin-mediated adhesion modulates signaling cascades in control of cell motility, survival, proliferation, and differentiation.

For many biological processes, most notably hemostasis and immunity, it is important that integrin-mediated adhesion can be regulated. The number of integrin-ligand bonds can be regulated through changes in cellular shape, lateral diffusion of integrins in the membrane, and integrin clustering; aspects that can be controlled through cytoskeletal organization. Additionally, the intrinsic affinity of individual integrins for their ligands can be regulated from within the cell, a process referred to as “inside-out signaling”.

Integrin-engagement triggers the formation of membrane extensions that are required for cell spreading on ECM surfaces, for migration of cells into sheets of other cells, or for engulfment of particles or pathogens by phagocytic cells. Ultimately, ligands, integrins, cytoskeletal proteins, and signaling molecules assemble in high local concentrations as aggregates on each side of the plasma membrane, forming “cell-matrix adhesions” in the case of integrins binding to ECM proteins. Integrin function largely depends on the connection of integrins to the cytoskeleton. The integrin cytoplasmic tails connect to the F-actin filaments through an exquisitely regulated multiprotein complex.

Integrin alpha 5 (CD49e, ITGA5) reference protein sequence may be accessed at Genbank, accession number NP_002196. The alpha chain is frequently paired with integrin β1, i.e. α₅β₁, which binds to an Arg-Gly-Asp (RGD) motif within fibronectin. The residues outside the RGD motif in fibronectin provide specificity as well as high affinity for the integrin-ligand pair. α₅β₁ integrin and Fn form a prototypic integrin-ligand pair, which mediates fibronectin fibril formation and governs extracellular matrix assembly, which is vital to cell function in vivo. Lack of α₅β₁ or Fn results in early embryonic lethality. In addition to the RGD sequence present in Fn type III module 10, a set of residues present in Fn type III module 9 (synergy site) contribute to high-affinity recognition by α₅β₁.

As used herein, an “antagonist,” or “inhibitor” agent refers to a molecule which, when interacting with (e.g., binding to) a target protein, decreases the amount or the duration of the effect of the biological activity of the target protein (e.g., interaction between leukocyte and endothelial cell in recruitment and trafficking). Antagonists may include proteins, nucleic acids, carbohydrates, antibodies, or any other molecules that decrease the effect of a protein. Unless otherwise specified, the term “antagonist” can be used interchangeably with “inhibitor” or “blocker”.

The term “agent” as used herein includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

Anti-integrin alpha 5 agent. As used herein, an anti-integrin alpha 5 (anti-α₅) agent blocks the activity of integrin alpha 5, particularly human integrin alpha 5. In some embodiments the anti-α₅ agent is an antibody that specifically binds to α₅, β₁, and/or α₅β₁ integrin. In some embodiments the anti-α₅ agent is a peptide or peptidomimetic, which may comprise an RGD motif. In some embodiments the anti-α₅ agent is a small molecule. In some embodiments an anti-α₅ agent blocks the binding of α5 and/or α₅β₁ to fibronectin. In some embodiments an anti-α₅ agent blocks the interaction of anti-α5 to β1 integrin.

Specific anti-α₅ agents of interest include, without limitation, humanized or chimeric versions of mouse anti-human CD49e antibodies: IIA (BD biosciences, function-blocking murine antibody); anti-human α5 (CD49e) Integrin: NKI-SAM-1; integrin alpha 5 beta 1 antibody M200 (Volociximab), a chimeric human IgG4 version of the murine IIA1 antibody; F200, the Fab derivative of a chimeric human IgG4 version of the alpha5beta1 function-blocking murine antibody IIA1; antibody PF-04605412, a fully human, Fc-engineered IgG1 monoclonal antibody targeting integrin α5β1 that blocks the attachment of the integrin to a substrate. Antibodies specific for human β1 integrin are also known in the art, including, for example, TS2/16, Poly6004, etc. U.S. Pat. No. 8,350,010, herein specifically incorporated by reference; teaches the small molecule peptidic inhibitor Ac-PHSCN-NH2 (disclosed in WO-9822617A1). ATN-161 is a five amino acid acetylated, amidated PHSCN peptide derived from the synergy region of human fibronectin PHSRN sequence. The arginine amino acid in the original sequence is replaced with cysteine residue. Analogs of ATN-161 include, for example, ATN-453, PHSCN-polylysine dendrimer (Ac-PHSCNGGK-MAP), PhScN (where histidine and cysteine were replaced with D-isomers), PHSC(S-OAc)N, PHSC(S-Me)N, PHSC(S-acm)N, which have been reported to be more potent than ATN-161.

The dosing and regimen for antibody administration, e.g. for safety profile, feasibility, activity, pharmacokinetic and pharmacodynamic behavior of an antibody such as volociximab, may follow the dosing utilized for cancer treatment, or may vary the dose for treatment of autoimmune disease. For example, dose levels may range from about 0.1 to about 25 mg/kg, administered daily, semi-weekly, weekly, every other week, monthly, etc. For delivery of an antibody such as Volociximab, the dosage for an adult human may be from about 0.1 mg/kg; from about 0.25 mg/kg; from about 0.5 mg/kg; from about 0.75 mg/kg; from about 1 mg/kg; from about 1.25 mg/kg; from about 2.5 mg/kg; from about 5 mg/kg; up to about 25 mg/kg, up to about 15 mg/kg; up to about 10 mg/kg. The total daily dose for an average human may be up to about 250 mg; may be up to about 200 mg; may be up to about 100 mg, may be up to about 75 mg, may be up to about 50 mg.

Antagonists of interest include antibodies as described above. Also included are soluble receptors, conjugates of receptors and Fc regions, and the like. Generally, as the term is utilized in the specification, “antibody” or “antibody moiety” is intended to include any polypeptide chain-containing molecular structure that has a specific shape which fits to and recognizes an epitope, where one or more non-covalent binding interactions stabilize the complex between the molecular structure and the epitope. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinantly, and may be modified to reduce their antigenicity.

Antibody fusion proteins may include one or more constant region domains, e.g. a soluble receptor-immunoglobulin chimera, refers to a chimeric molecule that combines a portion of the soluble adhesion molecule counterreceptor with an immunoglobulin sequence. The immunoglobulin sequence preferably, but not necessarily, is an immunoglobulin constant domain. The immunoglobulin moiety may be obtained from IgG1, IgG2, IgG3 or IgG4 subtypes, IgA, IgE, IgD or IgM, but preferably IgG1 or IgG3.

A straightforward immunoadhesin combines the binding region(s) of the “adhesin” protein with the hinge and Fc regions of an immunoglobulin heavy chain. Ordinarily nucleic acid encoding the soluble adhesion molecule will be fused C-terminally to nucleic acid encoding the N-terminus of an immunoglobulin constant domain sequence, however N-terminal fusions are also possible. Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion or binding characteristics.

Antibodies that have a reduced propensity to induce a violent or detrimental immune response in humans (such as anaphylactic shock), and which also exhibit a reduced propensity for priming an immune response which would prevent repeated dosage with the antibody therapeutic are preferred for use in the invention. These antibodies are preferred for all administrative routes, including intrathecal administration. Thus, humanized, chimeric, or xenogeneic human antibodies, which produce less of an immune response when administered to humans, are preferred for use in the present invention.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference). Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Alternatively, polyclonal or monoclonal antibodies may be produced from animals which have been genetically altered to produce human immunoglobulins, such as the Abgenix XenoMouse or the Medarex HuMAb® technology. Alternatively, single chain antibodies (Fv, as described below) can be produced from phage libraries containing human variable regions.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Small molecule agents encompass numerous chemical classes, though typically they are organic molecules, e.g. small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of GABA_{A} inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

Candidate antagonists can be tested for activity by any suitable standard means. As a first screen, the antibodies may be tested for binding against the adhesion molecule of interest. As a second screen, antibody candidates may be tested for binding to an appropriate cell line, e.g. leukocytes or endothelial cells, or to primary tumor tissue samples. For these screens, the candidate antibody may be labeled for detection (e.g., with fluorescein or another fluorescent moiety, or with an enzyme such as horseradish peroxidase). After selective binding to the target is established, the candidate antibody, or an antibody conjugate produced as described below, may be tested for appropriate activity, including the ability to block leukocyte recruitment to the central nervous system in an in vivo model, such as an appropriate mouse or rat epilepsy model, as described herein.

### Conditions for Treatment

Neurological inflammatory diseases. The term “inflammatory” response is the development of a humoral (antibody mediated) and/or a cellular (mediated by antigen-specific T cells or their secretion products) response. Inflammatory demyelinating diseases of the central nervous system are of particular interest and include, without limitation, multiple sclerosis (MS), neuromyelitis optica (NO), and experimental acquired encephalitis (EAE). Demyelinating inflammatory diseases of the peripheral nervous system include Guillain-Barre syndrome (GBS) with its subtypes acute inflammatory demyelinating polyradiculoneuropathy, acute motor axonal neuropathy, acute motor and sensory axonal neuropathy, Miller Fisher syndrome, and acute pandysautonomia; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy. Although not traditionally classified as an inflammatory disease, ALS has been found to have increased numbers of CD49e macrophages, and may be treated by the methods described herein.

Multiple sclerosis is characterized by various symptoms and signs of CNS dysfunction, with remissions and recurring exacerbations. Classifications of interest for analysis by the methods of the invention include relapsing remitting MS (RRMS), primary progressive MS (PPMS) and secondary progressive MS (SPMS). The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat can accentuate symptoms and signs.

The course is highly varied, unpredictable, and, in most patients, remittent. At first, months or years of remission can separate episodes, especially when the disease begins with retrobulbar optic neuritis. However, some patients have frequent attacks and are rapidly incapacitated; for a few the course can be rapidly progressive (primary progressive MS, PPMS), or secondary progressive multiple sclerosis (SPMS). Relapsing remitting MS (RR MS) is characterized clinically by relapses and remissions that occur over months to years, with partial or full recovery of neurological deficits between attacks. Such patients manifest approximately 1 attack, or relapse, per year. Over 10 to 20 years, approximately 50% of RR MS patients develop secondary progressive MS (SP MS) which is characterized by incomplete recovery between attacks and accumulation of neurologic deficits resulting in increasing disability.

Diagnosis is usually indirect, by deduction from clinical, radiographic (brain plaques on magnetic resonance [MR] scan), and to a lesser extent laboratory (oligoclonal bands on CSF analysis) features. Typical cases can usually be diagnosed confidently on clinical grounds. The diagnosis can be suspected after a first attack. Later, a history of remissions and exacerbations and clinical evidence of CNS lesions disseminated in more than one area are highly suggestive.

MRI, the most sensitive diagnostic imaging technique, can show plaques. It can also detect treatable nondemyelinating lesions at the junction of the spinal cord and medulla (eg, subarachnoid cyst, foramen magnum tumors) that occasionally cause a variable and fluctuating spectrum of motor and sensory symptoms, mimicking MS. Gadolinium-contrast enhancement can distinguish areas of active inflammation from older brain plaques. MS lesions can also be visible on contrast-enhanced CT scans; sensitivity can be increased by giving twice the iodine dose and delaying scanning (double-dose delayed CT scan).

Neuromyelitis optica (NMO), or Devic's disease, is an autoimmune, inflammatory disorder of the optic nerves and spinal cord. Although inflammation can affect the brain, the disorder is distinct from multiple sclerosis, having a different pattern of response to therapy, possibly a different pattern of autoantigens and involvement of different lymphocyte subsets.

The main symptoms of Devic's disease are loss of vision and spinal cord function. As for other etiologies of optic neuritis, the visual impairment usually manifests as decreased visual acuity, although visual field defects, or loss of color vision can occur in isolation or prior to formal loss of acuity. Spinal cord dysfunction can lead to muscle weakness, reduced sensation, or loss of bladder and bowel control. The damage in the spinal cord can range from inflammatory demyelination to necrotic damage of the white and grey matter. The inflammatory lesions in Devic's disease have been classified as type II lesions (complement mediated demyelinization), but they differ from MS pattern II lesions in their prominent perivascular distribution. Therefore, the pattern of inflammation is often quite distinct from that seen in MS.

Attacks are conventionally treated with short courses of high dosage intravenous corticosteroids such as methylprednisolone IV. When attacks progress or do not respond to corticosteroid treatment, plasmapheresis can be used. Commonly used immunosuppressant treatments include azathioprine (Imuran) plus prednisone, mycophenolate mofetil plus prednisone, Rituximab, Mitoxantrone, intravenous immunoglobulin (IVIG), and cyclophosphamide.

The disease can be monophasic, i.e. a single episode with permanent remission. However, at least 85% of patients have a relapsing form of the disease with repeated attacks of transverse myelitis and/or optic neuritis. In patients with the monophasic form the transverse myelitis and optic neuritis occur simultaneously or within days of each other. Patients with the relapsing form are more likely to have weeks or months between the initial attacks and to have better motor recovery after the initial transverse myelitis event. Relapses usually occur early with about 55% of patients having a relapse in the first year and 90% in the first 5 years. Unlike MS, Devic's disease rarely has a secondary progressive phase in which patients have increasing neurologic decline between attacks without remission. Instead, disabilities arise from the acute attacks.

Amyotrophic lateral sclerosis is a group of rare neurological diseases that mainly involve the nerve cells (neurons) responsible for controlling voluntary muscle movement. It is characterized by steady, relentless, progressive degeneration of corticospinal tracts, anterior horn cells, bulbar motor nuclei, or a combination. Symptoms vary in severity and may include muscle weakness and atrophy, fasciculations, emotional lability, and respiratory muscle weakness. Diagnosis involves nerve conduction studies, electromyography, and exclusion of other disorders via MRI and laboratory tests. Current treatment is supportive. The majority of ALS cases (90 percent or more) are considered sporadic.

Most patients with ALS present with random, asymmetric symptoms, consisting of cramps, weakness, and muscle atrophy of the hands (most commonly) or feet. Weakness progresses to the forearms, shoulders, and lower limbs. Fasciculations, spasticity, hyperactive deep tendon reflexes, extensor plantar reflexes, clumsiness, stiffness of movement, weight loss, fatigue, and difficulty controlling facial expression and tongue movements soon follow. Other symptoms include hoarseness, dysphagia, and slurred speech; because swallowing is difficult, salivation appears to increase, and patients tend to choke on liquids. Late in the disorder, a pseudobulbar affect occurs, with inappropriate, involuntary, and uncontrollable excesses of laughter or crying. Sensory systems, consciousness, cognition, voluntary eye movements, sexual function, and urinary and anal sphincters are usually spared. Death is usually caused by failure of the respiratory muscles; 50% of patients die within 3 yr of onset, 20% live 5 yr, and 10% live 10 yr. Survival for >30 yr is rare.

The drugs riluzole (Rilutek) and edaravone (Radicava) have been approved to treat certain forms of ALS, and may be provided in combination with an α5 integrin antagonist. Riluzole is believed to reduce damage to motor neurons by decreasing levels of glutamate, which transports messages between nerve cells and motor neurons. Clinical trials in people with ALS showed that riluzole prolongs survival by a few months, particularly in the bulbar form of the disease, but does not reverse the damage already done to motor neurons. Edaravone has been shown to slow the decline in clinical assessment of daily functioning in persons with ALS.

Animal models for ALS include mutations in the SOD1 gene. Missense mutations in the SOD1 gene on chromosome 21 were the first identified causes of autosomal dominant FALS. SOD1 is a ubiquitous cytoplasmic and mitochondrial enzyme which functions in a dimeric state to catalyse the breakdown of harmful reactive oxygen species (ROS), thereby preventing oxidative stress. Sod1^{-/-} mice do not have any motor neuron loss, but they have a significant distal motor axonopathy, demonstrating the important role of SOD1 in normal neuronal function. The significant loss of motor neurons in transgenic mice expressing mutant SOD1 is likely to result from a toxic gain-of-function.

The methods disclosed herein stabilize or reduce the clinical symptoms of MS, NMO, or ALS, e.g. by reducing the activity of CD49e+ monocytic cells in the central nervous system.

In an embodiment, methods are provided for enhancing removal of tattoos. Myeloid cells of the dermis are dominated by DT-sensitive, melanin-laden cells that correspond to macrophages that have ingested melanosomes from neighboring melanocytes. Those cells have been referred to as melanophages in humans. These melanophages are responsible for the capture and retention of tattoo pigment particles, which can undergo successive cycles of capture-release-recapture without any tattoo vanishing. By inhibiting macrophage activity through administration of an antagonist to CD49e, removal of undesired tattoos can be enhanced. The antagonist can be provided through a localized implant, intradermal injection, etc., or may be delivered systemically.

### Additional Agents

Statins are inhibitors of HMG-CoA reductase enzyme and may be provided in a combination therapy with an anti-α₅ agent, e.g. for the treatment of MS or NMO. Statins are described in detail, for example, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional compounds are disclosed in U.S. Pat. Nos. 5,208,258, 5,130,306, 5,116,870, 5,049,696, RE 36,481, and RE 36,520.

An effective dose of a statin is the dose that, when administered for a suitable period of time, usually at least about one week, and may be about two weeks, or more, up to a period of about 4 weeks, will evidence a reduction in the severity of the disease and/or control serum cholesterol levels. It will be understood by those of skill in the art that an initial dose may be administered for such periods of time, followed by maintenance doses, which, in some cases, will be at a reduced dosage.

The formulation and administration of statins is well known, and will generally follow conventional usage. The dosage required to treat autoimmune disease may be the same or may vary from the levels used for management of cholesterol in the absence of anti-α₅ agent treatment.

Statins can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. The formulation is optionally combined in a unit dose with an anti-α₅ agent.

Interferon beta is a drug in the interferon family used to treat multiple sclerosis (MS) and may be provided in a combination therapy with an anti-α₅ agent for treatment of MS. IFN-β1a is produced by mammalian cells while Interferon beta-1b is produced in modified E. coli. Interferons have been shown to have about a 18-38% reduction in the rate of MS relapses, and to slow the progression of disability in MS patients. Commercially available products include Avonex (Biogen Idec); Rebif (EMD Serono); and CinnoVex (CinnaGen). Closely related is Interferon beta-1b, which is marketed in the US as Betaseron, or Extavia.

Various formulations and dosages are conventionally utilized in the treatment of MS patients with IFN-β, which doses may be utilized in the combination treatments of the present invention, or may be utilized at a lower dose, e.g. 90% of the conventional dose, 80% of the conventional dose, 70% of the conventional dose, 60% of the conventional dose, 50% of the conventional dose, or less.

Avonex is sold in two formulations, a lyophilized powder requiring reconstitution and a pre-mixed liquid syringe kit; it is usually administered once per week via intramuscular injection at a dose of 30 μg. Rebif is administered via subcutaneous injection three times per week at a dose of 22 μg or 44 μg. Interferon beta-1b is usually administered at 250 μg on alternate days.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

A “subject” or “patient” in the context of the present teachings is generally a mammal. Mammals other than humans can be advantageously used as subjects that represent animal models of inflammation. A subject can be male or female.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis to, e.g., determine whether a subject is a responder or a non-responder to a therapy (e.g., an IFN treatment as described herein).

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

### Methods

The present disclosure provides methods for treating neurological inflammatory diseases, which may be a demyelinating autoimmune disease, such as multiple sclerosis. The methods comprise administering to the subject an effective amount of an agent that is an anti-α₅ agent as a single agent or combined with an additional one or more agents(s).

In certain embodiments the anti-α₅ agent is combined with a therapeutic dose of a statin. The active agents may be administered in separate formulations, or may be combined, e.g. in a unit dose. The formulation may be for oral administration. Optionally the anti-α₅ agent is combined as a single agent or with a statin in a combination with a second compound such as a cytokine; an antibody, e.g. tysabri; fingolimod (Gilenya); copaxone, etc. In some embodiments the cytokine is IFN-β.

In other embodiments an anti-α₅ agent may be combined with an agent, such as a cytokine; an antibody, e.g. tysabri; fingolimod (Gilenya); copaxone, etc., in the absence of a statin. In some embodiments, the patient is analyzed for responsiveness to cytokine therapy, where the selection of therapeutic agent is based on such analysis.

In some embodiments the combined therapies are administered concurrently, where the administered dose of any one of the compounds may be a conventional dose, or less than a conventional dose. In some embodiments the two therapies are phased, for example where one compound is initially provided as a single agent, e.g. as maintenance, and where the second compound is administered during a relapse, for example at or following the initiation of a relapse, at the peak of relapse, etc.

In various aspects and embodiments of the methods and compositions described herein, administering the therapeutic compositions can be effected or performed using any of the various methods and delivery systems known to those skilled in the art. The administering can be performed, for example, intravenously, orally, via implant, transmucosally, transdermally, intramuscularly, intrathecally, and subcutaneously. The delivery systems employ a number of routinely used pharmaceutical carriers.

In methods of use, an effective dose of an anti-α₅ agent of the invention is administered alone, or combined with additional active agents for the treatment of a condition as listed above. The effective dose may be from about 1 ng/kg weight, 10 ng/kg weight, 100 ng/kg weight, 1 μg/kg weight, 10 μg/kg weight, 25 μg/kg weight, 50 μg/kg weight, 100 μg/kg weight, 250 μg/kg weight, 500 μg/kg weight, 750 μg/kg weight, 1 mg/kg weight, 5 mg/kg weight, 10 mg/kg weight, 25 mg/kg weight, 50 mg/kg weight, 75 mg/kg weight, 100 mg/kg weight, 250 mg/kg weight, 500 mg/kg weight, 750 mg/kg weight, and the like. The dosage may be administered multiple times as needed, e.g. every 4 hours, every 6 hours, every 8 hours, every 12 hours, every 18 hours, daily, every 2 days, every 3 days, weekly, and the like. The dosage may be administered orally.

The compositions can be administered in a single dose, or in multiple doses, usually multiple doses over a period of time, e.g. daily, every-other day, weekly, semi-weekly, monthly etc. for a period of time sufficient to reduce severity of the inflammatory disease, which can comprise 1, 2, 3, 4, 6, 10, or more doses.

Determining a therapeutically or prophylactically effective amount of an agent according to the present methods can be done based on animal data using routine computational methods. The effective dose will depend at least in part on the route of administration.

### Pharmaceutical Compositions

The above-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the anti-α₅ agent is formulated with a second agent in a pharmaceutically acceptable excipient(s).

The subject formulations can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The anti-α₅ agent may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Example 1

### Single-Cell Analysis Reveals Differential Molecular Signatures in Myeloid Cells from Contrasting Models of Neuroinflammation Versus Neurodegeneration

Two polarities are the subject of much attention in brain pathology: neuroinflammation versus neurodegeneration. Here, we use single cell mass cytometry (CyToF) conducted with an unbiased data analysis to perform a system-wide analysis of the immune response in the R6/2 mouse model of Huntington's disease (HD), a neurodegenerative condition, versus the Experimental Autoimmune Encephalomyelitis (EAE) mouse model of Multiple Sclerosis (MS), the quintessential inflammatory disease of the brain. We identified three myeloid cell populations exclusive to the central nervous system (CNS), and present in both neuroinflammatory (EAE) and neurodegenerative (HD) conditions. Blood-derived monocytes, the counterpart of CNS-resident myeloid cells, consist of five subpopulations and were detected in EAE but were absent in HD. Single cell analysis revealed a vast disparity in signaling activity and cytokine production within similar myeloid populations in EAE compared to HD. In neuroinflammatory conditions, tightly organized signaling events occur in a stepwise manner, whereas these same signaling events are absent in neurodegenerative conditions. Furthermore, there is a notable difference in the cytokine profile at the single-cell level between these two neuropathologies, where multifunctional cells simultaneously secreting multiple cytokines correlated with neuroinflammation in EAE. These findings emphasize the differences in neuropathology between inflammatory and degenerative brain disease, and reveal selective therapeutic targets for these specific brain pathologies.

Two of the polarities in brain pathology, pit the concept of neuroinflammation in contrast to neurodegeneration. The cellular response in the former case is comprised of infiltration of peripheral adaptive and innate immune cells. In the latter, pathology is characterized by the activities of CNS-resident immune cells, namely, microglia and perivascular myeloid cells. In disorders, such as Huntington's disease (HD), as well as Alzheimer's disease (AD) or prion disease, there is little or no evidence for the entrance of the cells of the peripheral immune system within the CNS. This is in contrast to multiple sclerosis, acute disseminated encephalomyelitis, stroke and microbial infection, where there is rampant inflammation with migration of peripheral immune cells into the CNS. In MS, for example, blockade of the entry of peripheral immune cells to the brain with antibodies to key integrins has served as the mechanistic basis for the most potent approved therapy, approved now for a decade. However, in other neurological disorders including Alzheimer's disease, prion disease, amyotrophic lateral sclerosis (ALS), and Huntington's disease, there is no evidence of the same classical inflammatory response. Yet, in the contemporary literature, these neurodegenerative disorders are often referred to as neuroinflammatory or neuroimmune disorders.

Confusion in defining neuroinflammation versus neurodegeneration may arise from microgliosis—the proliferation and activation of microglia—which is a well-established hallmark of any insult to the CNS. Activation of microglia is accompanied by up-regulation and the release of a plethora of inflammatory mediators including chemokines and cytokines that are normally produced by cells of the peripheral immune system.

Refining the concept of neuroinflammatory versus neurodegenerative pathology is addressed here. In recent years, analyses of gene transcripts from bulk-processed samples identified several pathways that are implicated in CNS disease. One recent study compared inflammatory processes from a model of peripheral endotoxemia with models of neurodegenerative disease like Alzheimer's and ALS.

Here, we analyzed immune responses by using mass cytometry (CyTOF), allowing us to measure multiple parameters simultaneously in brain diseases at the single-cell level.

To this end, using mass cytometry (CyTOF) with an unbiased bioinformatic analysis of the data, we provide a system-wide view of the involvement of CNS-resident and blood-derived cell populations in two neurological disorders-experimental autoimmune encephalomyelitis and Huntington's Disease, which occupy different ends of the spectrum of neuroinflammation and neurodegeneration. We report differences in system-level signaling and cytokine production in these two polar examples of brain pathology, and help to clarify the vast differences in pathology in these two polarities of neuropathology.

### Results

Heterogeneous CNS-Resident Myeloid Populations.

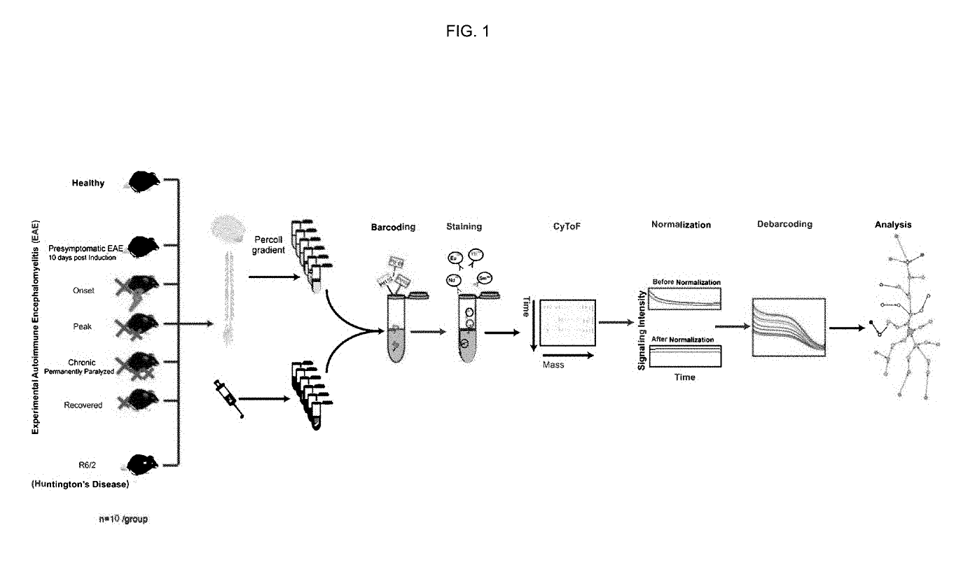

To investigate the immune response in neuroinflammatory and neurodegenerative conditions, we analyzed the cellular phenotype, the signaling properties, and the cytokine production in single-cell suspensions from the central nervous system (brain and spinal cord) and in the peripheral blood in examples of these two polar neuropathological conditions. We compared different clinical stages of experimental autoimmune encephalomyelitis (EAE), a model of neuroinflammatory disease resembling MS, with R6/2 transgenic mice, a model of Huntington's disease (HD), at the time the mice displayed tremor, irregular gait, abnormal movements and seizures, with single-cell mass cytometry (CyTOF)(FIG. 1).

In order to explore the phenotypic diversity of immune cell populations in the CNS and blood, we combined all the single cell datasets (all mice under all disease conditions for EAE, HD and healthy) and applied a population-mapping algorithm called X-shift. This algorithm was specifically developed to enable the discovery of rare cell populations in poorly characterized biological systems via nonparametric mapping of cell event density in multidimensional marker space. One of the most useful features of X-shift is that the algorithm automatically estimates the number of cell populations. Thus, the phenotypic space can be mapped automatically and, unlike most other single-cell clustering algorithms, this approach does not require user input. In order to visualize the phenotypic continuum of cell populations, output is organized into a Minimum Spanning Tree (MST), creating a 2-dimensional layout. Cell clusters are represented as nodes and are connected with edges and organized according to their overall phenotypic similarity based on the full panel of surface markers. Differences in cell frequency of each subpopulation across conditions are visualized by varying the size of each node proportionally to the frequency of the respective cluster in a given condition. Differences in marker expression levels across populations are visualized by coloring the nodes according to condition-specific marker expression levels. Visual inspection of node sizes and expression levels allowed us to identify lineage-specific groups within the MSTs and to depict the disease-specific cell populations.

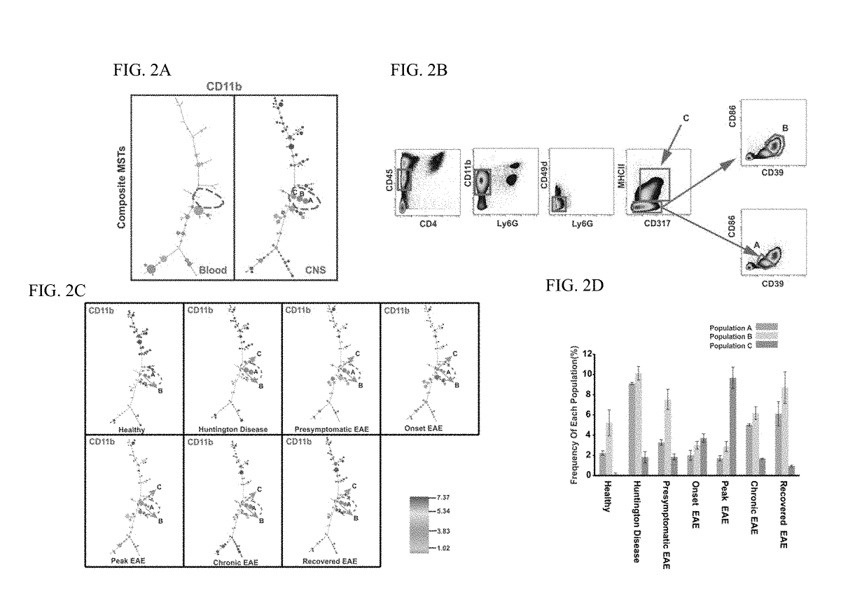

Comparisons of the composite MSTs for all blood samples with the composite MSTs from all CNS samples revealed three distinct subpopulations of CD11 b⁺ myeloid populations present in the CNS but absent in peripheral blood thereby identifying them as CNS-specific myeloid populations. These populations are defined here as population A, B, and C (FIG. 2a).

To deduce the sequence of gates that define the clustered populations of interest, we applied a feature of the X-shift algorithm called a Divisive Marker Tree (DMT) algorithm that automatically constructs an optimal marker-based classification of clusters. Setting the gates according to computationally defined thresholds we were able, by manual gating, to verify population A, B, and C, distinguishable by cell surface marker expression of CD45, CD11 b, CD317 (BST2/PDCA-1), major histocompatibility complex class II (MHCII), CD39, and CD86 (FIG. 2b).

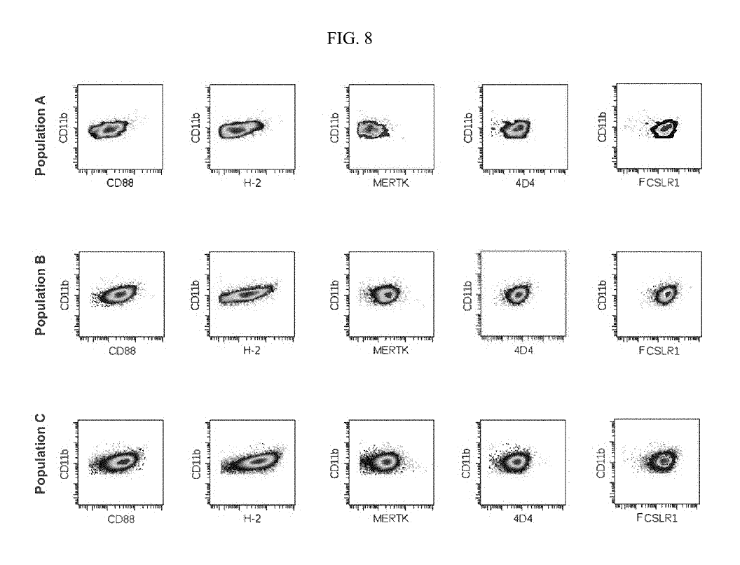

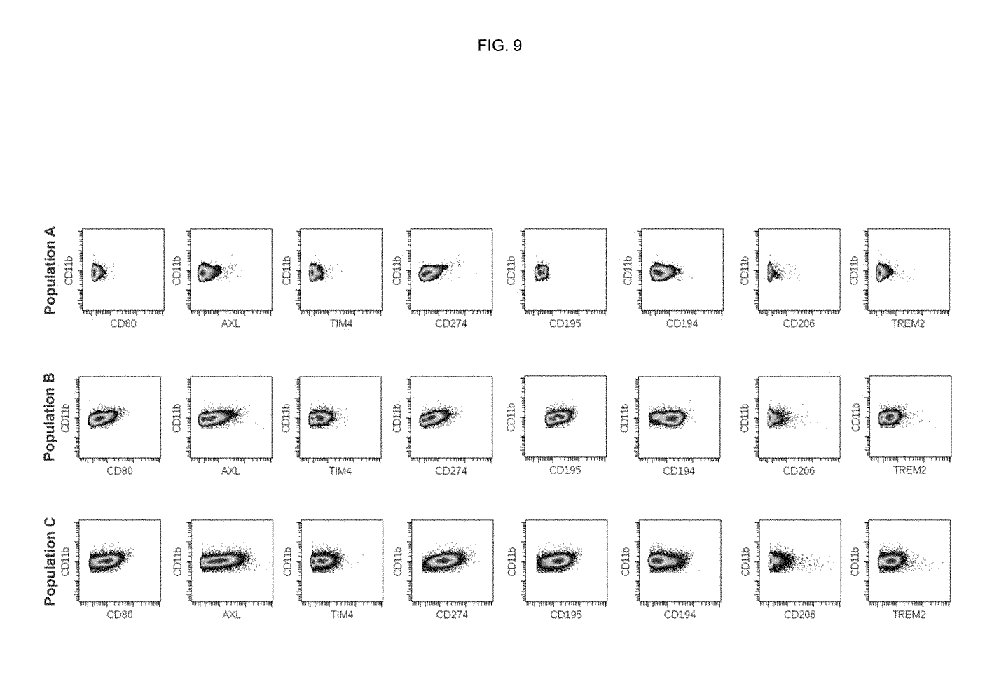

In addition to the main markers mentioned above which delineate the separation of each population, populations A, B, and C also expressed several other cell surface markers. Our analysis revealed that all three populations expressed low to medium levels of CD88, MHC class I (H2), TAM receptor tyrosine kinases Mer (MerTK), and the recently identified microglia markers 4D4 and fcrls. Populations A, B, and C lacked expression of lymphoid lineage markers such as CD3 (T cells), CD45R/B220 (B cells), monocyte markers (Ly6C), and granulocytic markers (Ly6G) (FIG. 8). These three CNS-specific populations were also characterized by the differential expression of a number of markers. Population B and C expressed different levels of CD80, TAM receptor Axl, T-cell immunoglobulin mucin protein 4 (TIM4), D274 (PD-L1), CD195 (CCR5), CD194 (CCR4), and low levels of CD206 and TREM2, while population A lacked the expression of all these markers (FIG. 9). The expression level of these markers changed depending on disease conditions.

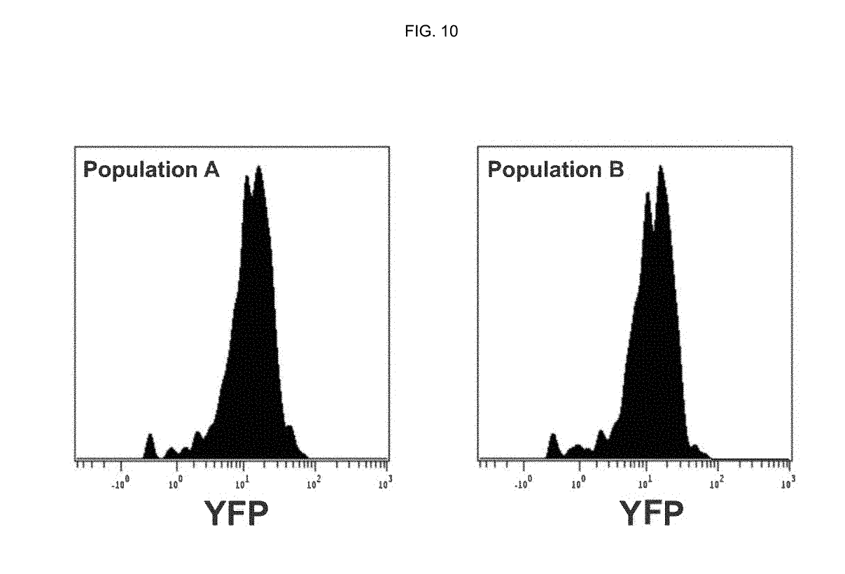

There is a lack of consensus for a specific marker distinguishing CNS-resident myeloid cells—microglia—from peripheral blood-derived macrophages. With the emergence of new antibodies and a transgenic mouse model, however, distinctions have been made between CNS-resident myeloid cells and infiltrating myeloid populations. Here, we defined these three populations (A, B, and C) as CNS-resident myeloid cells based on their presence in only the CNS (not in peripheral blood) coupled with the expression of phenotypic markers, low CD45-traditionally believed to mark microglia in the CNS—and Fcrls. We confirmed this possibility using conditional Cx3cr1^{creER} Rosa26-YFP mice that express YFP after tamoxifen administration. The persisting YFP raises the possibility of identifying microglia and other long-lived macrophages while YFP disappears in short-lived cells, e.g. peripheral monocytes. Here, we were able to identify these three populations in conditional Cx3cr1^{creER} Rosa26-YFP mice and confirm that they express YFP (FIG. 10). In this paper, for the sake of simplicity, we avoid calling them microglia and refer to them as CNS-resident myeloid cells, which could comprise microglia, meningial macrophages, and perivascular macrophages. Taken together, this multi-parameter analysis provided a high-resolution view of the phenotypic heterogeneity that exists within the CNS-resident myeloid population.

Neuroinflammatory and Neurodegenerative Conditions Mark Congruent CNS Myeloid Cell Populations.

To investigate whether disease-specific cues modulate the presence and the frequency of three CNS-resident myeloid cells, we analyzed the MSTs and confirmed the findings by manually gating, in all biological replicates of healthy, HD as well as five different states of EAE: presymptomatic, onset, peak, chronic, and recovered (FIG. 2c,d).

Cell frequency analysis and representative nodes in the MST in independent biological replicates of each disease state demonstrated that all three populations were altered in association with the disease states (FIG. 2c,d). Notably, the presence of all three CNS-resident myeloid populations was present in both the neurodegenerative and neuroinflammatory conditions. These data reinforce conclusions from previous studies that suggest neurodegenerative and neuroinflammatory conditions provoke a similar “immune response” since, at a first glance, similar populations are indeed observed.

Subpopulation C was elicited by both EAE and HD disease conditions and barely detectable in a healthy CNS (frequency of 0.1%). In EAE mice subpopulation C continued to expand from the presymptomatic stage (frequency of 1.8%) to the peak of disease (frequency 9.7%). Thereafter, the frequency of subpopulation C declined in chronic EAE animals with permanent paralysis and in recovered EAE mice (0.9% and 1.7% respectively) (FIG. 2d). Chronic EAE has long been considered to resemble the progressive forms of MS, which are categorized as the neurodegenerative aspects of the disease.

Distinct Signaling Phenotypes in CNS Myeloid Cells in Neuroinflammatory Versus Neurodegenerative Conditions.

While the above analysis of cell frequencies suggested similarities in both neuroinflammatory and neurodegenerative conditions, an analysis of signaling pathways, as discussed below, revealed differences in various key parameters including cell signaling and cytokine production.

To parse differences in signaling in population A, B, and C, we simultaneously compared the intracellular signaling behavior at different stages of EAE as well as Huntington's disease. To examine this, we analyzed the abundance of phosphorylated signal transducers and activators of transcription (STAT) 1, 3, 5, cAMP response element-binding protein (CREB), MAP kinase-activated protein kinase 2 (MAPKAPK2), nuclear factor-kappa B (NF-κB (p65)), CCAAT/enhancer-binding protein alpha and beta (C/EBPα, C/EBPβ) proteins. Analysis of these signaling pathways revealed three areas of interest.

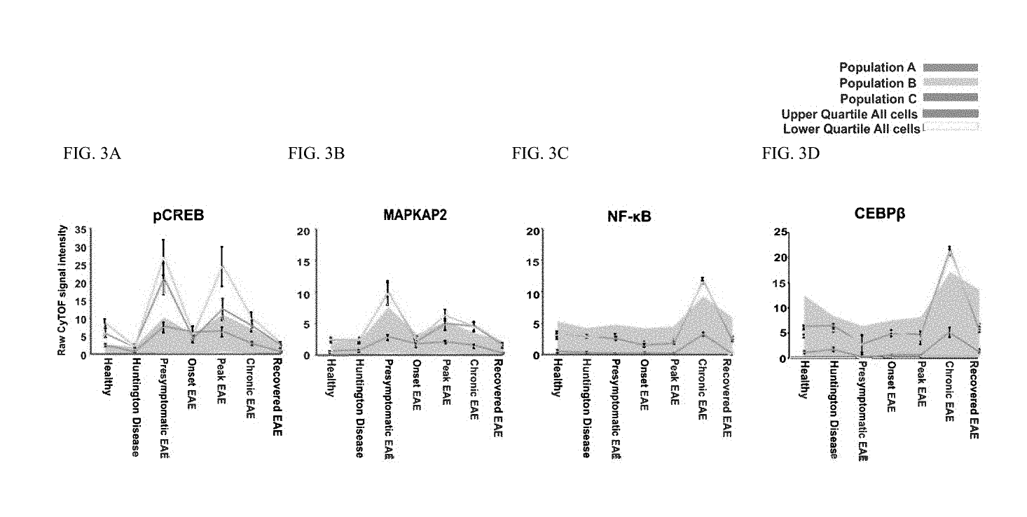

First, there are substantial differences in the expression patterns of these signaling proteins across all of the three CNS myeloid subsets, where population B and C showed a high level of signaling, but population A differed substantially from these two subsets with a very low expression level of signaling proteins (FIG. 3a-d), potentially reflecting a different functional role for each of these populations.

Second, this analysis identified that the development and progression of the inflammatory response in the CNS in populations B and C during the development of EAE is a tightly orchestrated process involving a key inflammatory signaling pathways in sequence. In the presymptomatic stage of EAE—where no clinical signs of disease have been developed in mice yet—a significant increased level of pCREB and pMAPKAPK2 expression represents the only signaling signature in population B and C (more than a 3-fold and 6-fold increase compared to healthy mice respectively) (FIG. 3a, b). At the peak of EAE disease a second wave of increased expression of pCREB and pMAPKAPK2 in population B and C emerged as a signaling hallmark (FIG. 3a, b) similar to what we observed in the presymptomatic stage and in agreement with previous studies. Interestingly, in chronic EAE—where animals never recovered from paralysis—up regulation of NF-κB(p65) in concert with C/EBPβ in population B and C were identified as the only players of a signaling cascade (FIG. 3c,d). These data indicate that in EAE there is a sequence of inflammatory signaling steps.

Lastly, these inflammatory signaling hallmarks were noticeably absent in population A, B, and C in HD compared to EAE (FIG. 3a-d) suggesting considerable differences in signaling properties in neurodegenerative conditions (HD) compared to neuroinflammatory conditions (EAE) in CNS-resident myeloid cell populations.

While similar CNS-resident myeloid cell populations were identified in both neuroinflammatory and neurodegenerative conditions, the nature of the signaling properties under these conditions were noticeably different suggesting a different functional capacity for these cells in each disease condition.

Multiple Cytokine Producing Myeloid Cells in Neuroinflammation Versus Neurodegeneration.

To gain a more comprehensive understanding of what cytokines are synthesized in EAE versus HD, we evaluated the in vivo cytokine production by these defined populations of myeloid cells. We avoided any ex vivo stimulation and used only a protein transporter inhibitor to avoid the secretion of cytokines (see material and methods). To test whether any of the identified populations have the capability of cytokine production, we adapted CyTOF technology to quantify a panel of eight synthesized cytokines: tumor necrosis factor-α (TNF-α), interferon-γ (IFN-γ), IFN-β, interleukin-10, IL-6, IL-17A, granulocyte-macrophage colony-stimulating factor (GM-CSF), and transforming growth factor-β (TGF-β) at the single-cell level. Each subpopulation was hand gated according to the criteria defined above (see FIG. 2b). We calculated the fraction of cells detected to secrete a given cytokine, defined by expression values exceeding the 90th percentile of a healthy sample for each cluster.

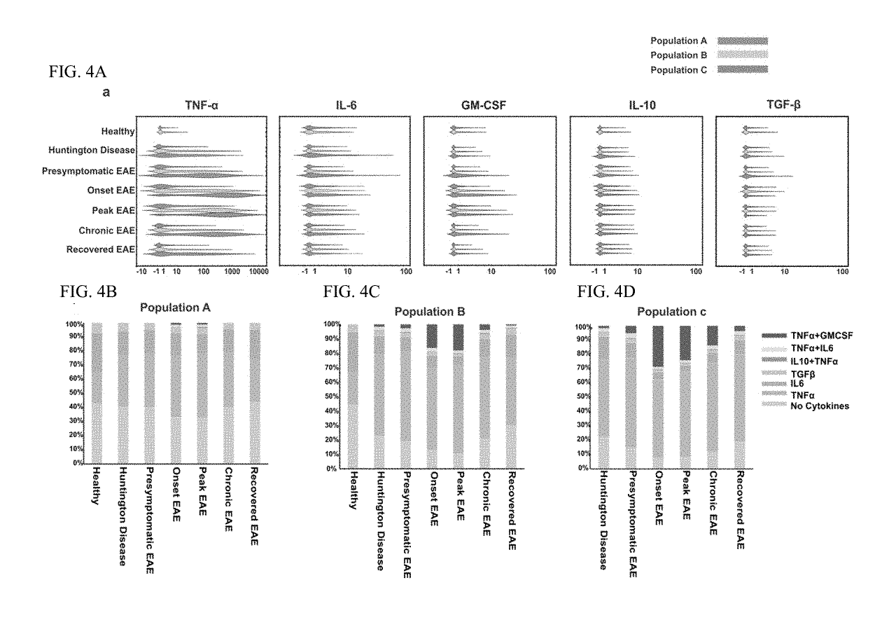

Among the eight cytokines evaluated, TNF-α was the most prominently produced cytokine in the three identified CNS-resident myeloid populations (A, B, C) where the percentage of TNF-α expressing cells increased significantly under both neuroinflammatory and neurodegenerative conditions compared to healthy cells (FIG. 4a). Most notably, in population B and C during different clinical scores of EAE disease—presymptomatic, onset, peak, and in the case of population C, chronic—the majority of cells (up to 80%) produced TNF-α whereas the percentage of TNF-α expressing cells ranged from 30%-50% in the neurodegenerative model (HD). In addition to TNF-α, a modest percentage of cells in these three populations expressed GM-CSF, IL-6, IL-10, and TGF-β (FIG. 4a).

Recent single cell studies suggest that there is significant heterogeneity among the single cell cytokine signatures of each given cell population. To exploit the multifunctional nature of each population at a single-cell level, we subsequently applied the X-shift clustering algorithm. Each population was clustered based on expression patterns of cytokines only, and the frequency of cells that produce each cytokine alone or in any combination at the single-cell level in each disease condition was assessed. Interestingly, a high level of functional heterogeneity in terms of the pattern of cytokine expression was identified within each population, which is defined as relatively homogeneous when cell surface markers are the only criteria for clustering.

Seven distinct subsets of cytokine-producing cells were delineated in populations A, B, and C at the single-cell level based on producing TNF-α, IL-6, TGF-β, and a combination of TNF-α with IL-6, GM-CSF, IL-10 or the lack of cytokine production (FIG. 4b). The frequency and the patterns of cytokine production of these distinct subsets differed directly in correlation to each disease state.

Quantifying the fraction of each of these seven identified subsets in each population and different disease conditions, we found that, in a healthy state, cells produced either a single cytokine or no cytokine at all, with most (42-44%) of the cells producing no cytokines (FIG. 4b). The frequency of single-positive TNF-α-producing cells increased significantly in comparison to the healthy state in both neuroinflammatory and neurodegenerative conditions whereas the frequency of IL-6 and TGF-β-producing cells decreased (FIG. 4b).

The disease conditions prompted the emergence of three multifunctional subsets that are clearly identifiable: dual TNF-α and GM-CSF producing cells, dual TNF-α and IL-10-producing cells, and dual TNF-α and IL-6-producing cells (FIG. 4b). Most noticeably, the frequency of GM-CSF and TNF-α co-expressing subset in populations B and C significantly increased during neuroinflammatory conditions especially at the onset and peak of EAE disease making this subset the second most abundant subset among cytokine-producing cells (up to 18% and 29% respectively) (FIG. 4b). Conversely, in neurodegenerative conditions, the frequency of this subset was very low—0% to 2%—in all three populations. With respect to other multifunctional subsets, both neuroinflammatory and neurodegenerative conditions also elicited the emergence of a low frequency of TNF-α⁺ IL-6⁺ and TNF-α⁺ IL-10⁺ multifunctional cells (2-3%). By comparing the cytokine profile in neuroinflammatory and neurodegenerative conditions, then, we can identify the GM-CSF, TNF-α dual producing subset as one of the defining signatures of neuroinflammatory conditions (FIG. 4b).

Moreover, among the three CNS-resident populations (A, B, and C), in population A, in contrast to the other two populations, a significant fraction of cells produced no cytokines in healthy and disease conditions, and the cytokine producing subsets were dominated by single cytokine producing cells even during disease conditions with multi-functional subsets comprising a very small percentage of cells (only 1%) (FIG. 4b). This result is important as the analysis of signaling properties of this population, as represented above, showed that population A has a lower expression level of signaling molecules compared to the other two populations (FIG. 4b).

Together, these data highlight a fundamental property of three identified CNS-resident myeloid cell populations, by demonstrating that each population, which is defined as relatively homogeneous by cell surface markers, in fact, contains heterogeneous functional subsets based on their cytokine secretion profile. Response to either inflammation or to degeneration skews the cytokine profile of each population towards an increase and drives the development of multifunctional subsets that produce two cytokines simultaneously. Although both neuroinflammatory and neurodegenerative conditions elicited the development of double positive TNF-α, GM-CSF producing cells, the high frequency of this subset correlated best with the height of neuroinflammatory conditions in EAE—peak and onset—in two populations (B and C). Populations B and C demonstrated pronounced inflammatory signaling properties, as well. The frequency of cells in these subsets was extremely low or was not observed, however, in pathologies such as HD, or in population A (in either HD or EAE) which had very low inflammatory signaling properties.

Blood-Derived Monocyte Subsets Exhibit Different Kinetics of Migration to CNS in Inflammatory Versus Degenerative States.

In the paradigm of classical inflammation the inflammatory response is defined by the activation of tissue-resident macrophages as the first line of defense and the subsequent recruitment of leukocytes from the blood into the affected tissue. Prominent in this cascade is the migration of monocytes into peripheral tissues to contribute to the inflammatory process and to replenish the resident tissue macrophages. In some cases, these monocytes disappear without contributing to the pool of tissue-resident macrophages. Like inflammation in peripheral tissues, monocyte infiltration has been linked to inflammatory responses in diseases of the central nervous system. For example, blood-derived macrophages exacerbate EAE pathology; however, they do not contribute to inflammation in neurodegenerative diseases.

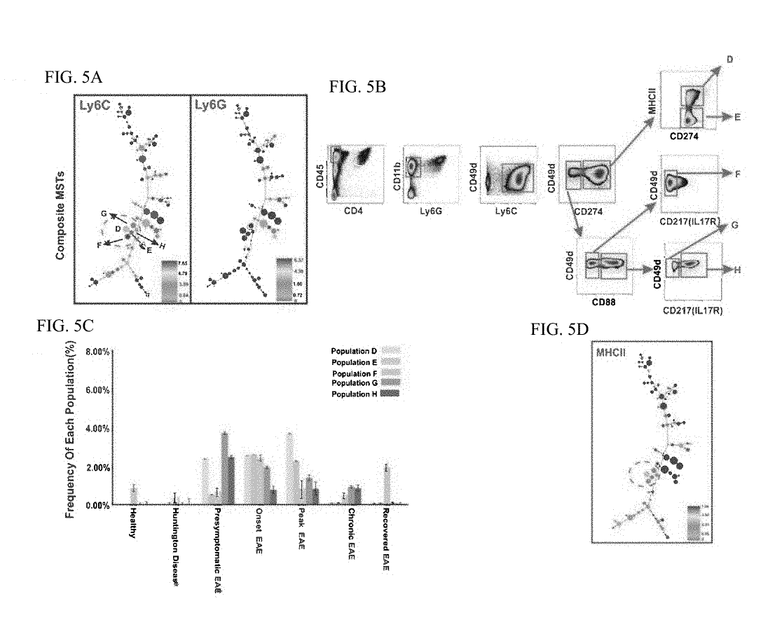

Since a significant part of the inflammatory response in the CNS is due to the entry of peripherally-derived myeloid cells, we next characterized the properties of these cells under neuroinflammatory (EAE) and neurodegenerative conditions (HD). Monocytes were distinguished from other myeloid cells (CD11b+ cells) based on expression of their key surface marker Ly6C and lack of Ly6G expression. A composite minimum spanning tree (MST) from all samples combined revealed five discrete Ly6C⁺Ly6G⁻ cell clusters in CNS samples (FIG. 5a). The X-shift algorithm separated the Ly6C compartment into five separate clusters (D, E, F, G, and H), and the Divisive Marker Tree visualization revealed that the main markers driving the separation are CD274 (PD-L1), CD88, IL-17R, and MHCII (FIG. 5b). To understand the relative contribution of circulating monocytes to the immune-cell heterogeneity in the CNS, we analyzed the frequency of each of these five monocyte subsets in the healthy state and under different clinical stages of neuroinflammation and neurodegeneration (FIG. 5c). Analyzing the frequency of each of these five subsets in the CNS of healthy animals and in different phases of EAE and HD indicated a selective recruitment of each of these monocyte subsets in different disease conditions (FIG. 5c). The most striking difference between neuroinflammatory and neurodegenerative conditions is that, in agreement with previous studies, we observed no contribution of monocytes (an average of less than 0.4%) in the CNS in the neurodegenerative condition HD. Of note also, and in accordance with earlier reports, in healthy and recovered CNS, similar to HD, there is a very low frequency of monocytes (0.8% to 1.2% respectively) and only one of the identified populations—population F—was detected. In contrast, inflammatory stages of EAE—presymptomatic, onset, and peak—evoked the presence of all five identified monocyte subsets (FIG. 5c). In chronic EAE we observed a low frequency (0.5 to 0.9%) of three out of five identified monocyte subsets (FIG. 5c).

An emerging theme from these data, in concert with our previous findings and those of others, is that the significant recruitment of monocytes is a transient and inflammatory-driven event. Once inflammation disappears, or is significantly diminished, monocytes largely vanish. The image of monocytes as the key player that triggers the progress of the disease to paralytic stage in EAE, a concept put forward by our own previous studies and others, now becomes more nuanced given our discovery of the considerable heterogeneity of this cell population.

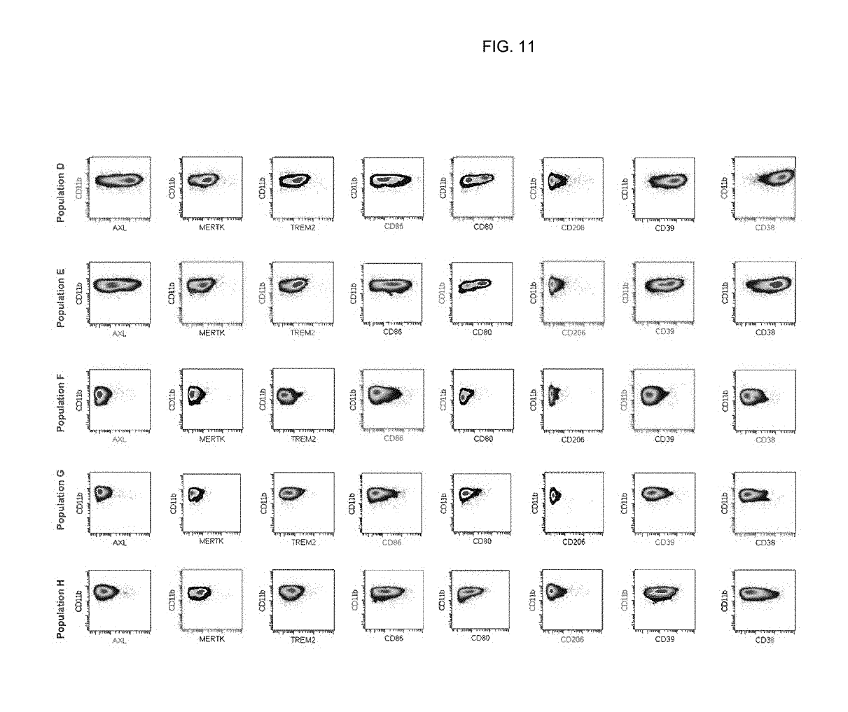

To gain a detailed understanding of how these various monocyte subsets contribute to inflammation in different disease states, we compared their phenotype and functional profiles to determine whether there any appreciable difference. We found that costimulatory molecules (CD80, CD86), receptors involved in purinergic signaling (CD38, CD39), phagocytic receptor for apoptotic cells like the TAM receptor tyrosine kinases Mer, Axl and the mannose receptor CD206 as well as TREM2 were up-regulated in population D and E while both population F and G expressed low levels of these markers and population H expressed a medium level (FIG. 11). In line with their expression of co-stimulatory molecules (CD80, CD86), the expression of MHC class II in population D and E (FIG. 5d) further suggests an antigen presenting function in the Ly6C⁺ compartment. Moreover, population D and E are only detected in the presymptomatic, onset, and peak phases of EAE and their number increased with the progression of the disease from the presymptomatic to peak stage. Conversely, these two populations were absent in chronic and recovered EAE as well as in healthy animals and HD (FIG. 5c). Considering the timing of their occurrence and the fact that they are only observed in T cell-mediated conditions such as EAE, and not in the neurodegenerative condition HD, these two subsets are potentially responsible for the activation of antigen specific T cells in EAE.

Differential Expression of Cell Surface Phenotype on Infiltrating Versus Resident Myeloid Cells Reveals Therapeutic Targets.

Microglia and peripheral-derived myeloid cells have distinct developmental origins, renewal mechanisms, and exert different functions in pathological processes even though they share similar morphology and major lineage cell surface markers. We explored these different cell types in reference to phenotypic surface proteins and functional markers—such as signaling and cytokines.

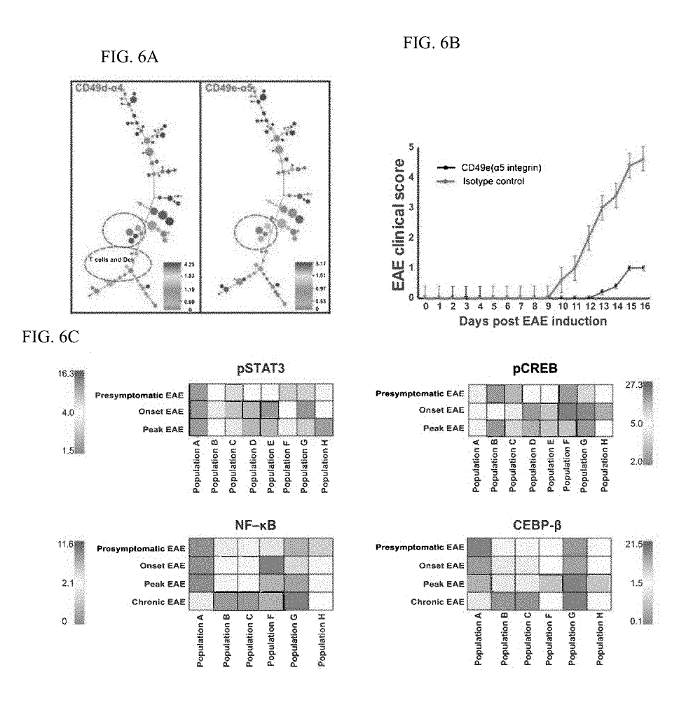

Comparing the cell surface markers in identified CNS-resident myeloid cell populations (A, B, C) with identified monocyte populations (D, E, F, G, H), we observed that the expression of adhesion molecules CD49d (α4 integrin) and CD49e (α5 integrin) were only present in blood-derived myeloid populations and not in CNS-resident myeloid cell populations (FIG. 6a). While CD49d (α4 integrin) was also expressed in other blood-derived populations such as T cells, DCs and granulocytes clusters, CD49e was only expressed by Ly6C⁺ subpopulations (FIG. 6a). CD49e binds fibronectin, an extra cellular matrix glycoprotein that is deposited in multiple sclerosis lesions, particularly around blood vessels. The expression of CD49e on monocytes suggests that CD49e-fibronectin interaction promotes migration of these cells to the CNS parenchyma.

To investigate if interfering with the entry of monocytes into the CNS by blocking their entry will affect the course of EAE disease, we treated EAE mice with MFR5 antibody specific to CD49e or its isotype as a control. The onset of the disease in mice treated with anti-CD49e antibody was significantly delayed compared with control group. Markedly, antibody treatment reduced the severity of the disease and the animals never reached to paralytic stage (FIG. 6b).

Blocking the homing of T lymphocytes and monocytes to the CNS using an antibody specific for α4 integrin suppressed EAE and reduced relapse rates in MS patients. Unfortunately, in a subset of individuals, this treatment leads to the reactivation of viral infections and progressive multi focal leukoencephalopathy. Lack of CD49e (α5 integrin) expression on T cells and its ability to reduce the severity of the disease in EAE, provides a rationale for a therapeutic strategy that specifically targets monocyte entry. Such a strategy might have potentially fewer side effects than existing therapies.

Discrepancies in Expression of Signaling Properties and Cytokine Profiles on Infiltrating Versus Resident Myeloid Cells.

Our earlier findings and others suggest evidence of functional differences between the blood-derived macrophages and CNS-resident myeloid cells during CNS inflammation. We next determined if the monocyte populations had different or similar signaling states in response to the same disease conditions compared to the CNS-resident myeloid cell populations in order to identify the mechanisms underlying their reported functional differences. A comparison of the relative expression of signaling molecules across the different populations of these two cell types confirmed that several signaling proteins were differentially expressed under the same disease conditions (FIG. 6c).

Expression of pSTAT3 was higher in several monocyte populations at the onset (population D and E) and peak (population D, E, and H) of EAE compared to all three CNS-resident myeloid cell populations (FIG. 6c). An increase in the transcription factor pSTAT3 is recognized as an important mediator of inflammation in MS patients.

In contrast, pCREB expression was markedly higher in CNS-resident myeloid cells, particularly population B and C in relation to monocyte populations (FIG. 6c) supporting a fundamental difference between infiltrating monocytes when compared to resident CNS-resident myeloid cells. The proliferation of CNS-resident myeloid cells but not monocytes, and the up-regulation of proliferation-related genes such as fos during the course of EAE in CNS-resident myeloid cells, has recently been reported. CREB is the main transcriptional regulator of the fos gene. The present results demonstrating pCREB expression are concordant with patterns of microglial proliferation and fos expression, and suggest that CREB pathways promote proliferation of CNS-resident myeloid cells during EAE. NF-κB and C/EBPβ expression were also increased in CNS-resident myeloid cell populations but not monocyte populations during EAE disease (FIG. 6c).

These studies support a model for signaling behavior of myeloid cells involved in the pathology of EAE disease; in presymptomatic stages, CNS-resident myeloid cells are the principal participants with pCREB and MAPKAPK2 upregulation as their signaling signature. At the onset of clinical disease signaling pathways switch to blood-derived myeloid cells, exhibiting their major signaling response with pSTAT3. At the peak of the disease, both cell types are involved in the signaling response but have different phenotypes, with CNS resident myeloid cells mainly up-regulating pCREB and MAPKAPK2 and monocytes up-regulating pSTAT3. In chronic disease, the signaling switches back to CNS-resident myeloid cells with expression of NF-κB and C/EBPβ during the chronic phase of EAE.

The difference in signaling responses of the CNS-resident myeloid cell populations, elicited by the same disease conditions, compared to monocyte populations, may explain their disparate effector properties during different stages of inflammation. On the basis of these results, we hypothesized that different phenotypes (FIG. 6a) and signaling properties (FIG. 6c) of CNS-resident myeloid cells and infiltrating monocytes should be reflected in distinct cytokine expression profiles during EAE pathology.

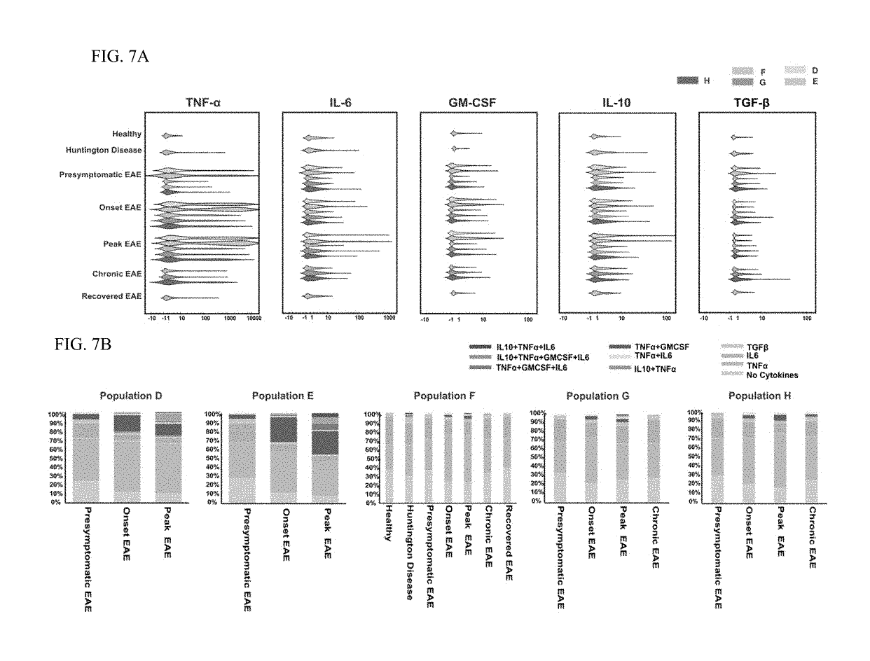

Therefore, we next assessed the cytokine production capacity of each of the monocyte populations, using the same method as described above in CNS-resident myeloid cells populations, by manual gating each monocyte population in our cytokine assay. Monocyte and CNS-resident myeloid cell populations had similar cytokine expression profiles, predominantly producing TNF-α followed by IL-6, GM-CSF, IL-10, and TGF-β (FIG. 7a). However, since this global analysis masks the heterogeneity within each population at the single-cell level based on any combination of cytokines, we next analyzed the profile of multiple cytokines produced by single cell populations using the X-shift clustering algorithm. Each population was clustered based on expression patterns of cytokines only. Comparative analysis of the five monocyte populations with the three CNS-resident myeloid cell populations revealed that in addition to seven distinct populations of cytokine-producing cells that were identified in CNS-resident myeloid cell populations (FIG. 4b-d), some of the monocyte populations have three additional multiple-cytokine-producing subsets in EAE (FIG. 7b). These three new multifunctional subsets consisted of triple cytokine producer cells, TNF-α⁺GM-CSF⁺IL-6⁺ and TNF-α⁺IL-6⁺ IL-10⁺, and quadruple cytokine producing cells, TNF-α⁺GM-CSF⁺IL-6⁺IL-10⁺ (FIG. 7b), whereas multifunctional subsets in microglia populations were only double positive (FIG. 4b-d). These three subsets were only identified at the onset and peak of EAE and had a significantly higher frequency at the peak of the disease compared to the onset (FIG. 7b). Therefore, although both CNS-resident myeloid cells and monocyte populations produced similar cytokines, there was a marked difference at the single cell level in the cytokine production profile of these two cell types elicited by the same disease stimuli.

Here we challenge a prevailing view where cellular and molecular activation across various neuropathologic conditions is routinely labeled “neuroinflammation”, despite striking differences in how these conditions appear under the microscope and how they present clinically. We analyzed two distinct polarities in CNS pathology, EAE and Huntington's disease, at a single cell level with mass cytometry, and made several stark observations. First, the details of the molecular response in these two pathologies in CNS-resident myeloid cells are quite different across many features including the biochemical signaling pathways that are activated, and the cytokines that are produced. Activation of these resident myeloid cells should not, therefore, be referred to with blanket descriptions such as “inflammatory” or “immune”. Second, CNS-resident myeloid cells and their peripherally derived myeloid counterparts have divergent molecular responses under these two pathologic conditions in the CNS.

The cellular and molecular roadmap defining inflammation outside the brain, in the so-called periphery (outside the blood brain barrier), is comprised of three features: an elevation in certain cytokines and chemokines, activation of tissue-resident macrophages, and recruitment of leukocytes from peripheral blood to the site of injury in the brain, resulting in local tissue pathology. However, the definition of inflammation in diseases of the CNS is controversial.

For the past two decades, the term neuroinflammation, referring to inflammation within the CNS, has signified any cascade of cellular and molecular reactions that are observed with diseases or injury of the CNS. This oversimplification, unfortunately, has led to assignment of the same cellular pathophysiology for neurodegenerative conditions and for neuroinflammatory diseases. One of the consequences is that similar therapeutic approaches have been suggested as putative treatments for widely disparate pathologies.

While MS, the quintessential and most prevalent inflammatory disease of the brain, features a rather “classic” immune reaction with aspects of innate and adaptive inflammation in the brain, the pathology in neurodegenerative diseases involves entirely different pathologic elements, primarily activation and proliferation of CNS-resident cells, including microglia, and perivascular myeloid cells and the release of cytokines and chemokines without the involvement of adaptive humoral or cellular immune responses. Yet, microglia activation and the detection of elevated levels of cytokines in the brain does not induce migration of peripheral immune cells to the brain, nor does it induce adaptive immunity in the brain. Microglial activation in itself should therefore not be used to categorize a disease as having a neuroinflammatory response.

In fact, numerous studies describe the presence of cytokines as well as activated CNS-resident myeloid cells in the absence of any pathology during the early development and adult brain where they both play a necessary function in neurogenesis, synaptic plasticity, and hemostasis. Such findings in a normal developing brain are not indicative of an immune response.

Here with an unbiased data-driven approach, we identified three CNS-specific myeloid populations (A, B, C) in both EAE and HD models. These populations increased in total frequency under both pathologies, EAE and HD. This result provides at least some basis for the contention that different CNS diseases involving microglia have “similarities”. Whether these similarities are sufficient to allow disparate pathologies to be called “neuroinflammatory” is problematic. Activation of CNS-resident myeloid cells in any pathology should not be benchmarked as an immune response.

Here we show that three CNS-resident myeloid populations in HD displayed highly discordant signaling properties when compared to their counterparts at different clinical stages of EAE, where conventional inflammation is present in the brain. In EAE, two of the CNS-resident myeloid populations developed a closely coordinated series of signaling events with pCREB and MAPKAPK2 as the signature for signaling during the presymptomatic stage of disease and prior to clinical paralysis, and at the peak of disease when paralysis is manifest, whereas both NF-κB and C/EBPβ signaling pathways characterized the chronic state. By contrast, these populations in HD samples with clinical disease did not exhibit any major expression of these signaling pathways contrary to previous reports. In particular, the lack of similarity in signaling activity between HD and chronic stage EAE, where mice in both models developed permanent functional impairment, is notable. Chronic EAE, or the secondary progressive phase of MS, has repeatedly been described as the “neurodegenerative” phase of MS in literature.

Our results, showing NF-κB and C/EBPβ signaling in CNS-resident myeloid cells in chronic EAE, and the lack therein of any such signaling activity in HD, emphasizes that although chronic EAE and HD are both categorized as neurodegenerative conditions, the nature of the pathologic response in them is divergent.

The difference in the functional properties of CNS-resident myeloid cells in the HD model compared to MS models was also reflected in their respective profiles of cytokine secretion. While, from an analysis of the total population, these three populations in healthy and both disease conditions demonstrated the ability to generate similar cytokines—albeit with different frequencies—analysis at the single-cell level confirmed that each population, in fact, contains different subsets based on their cytokine production profiles. Moreover, these subsets are altered in divergent ways in the polar disease conditions.

The striking difference between MS and HD models was the surge of cells that secrete multiple cytokines in EAE—TNF-α and GM-CSF, for example. Such dual secretors constituted a substantial portion of the total cytokine producing cells in onset and peak of the disease. These findings indicate that each cell within a subset purified on the basis of cell surface markers, may have a nuanced cytokine profile. Analysis of cytokine levels as a marker of immune response might be interpreted in the context of whether the cells are secreting single or multiple cytokines.

Establishing the extent and role of blood-derived myeloid cells over the course of disease in different neurological conditions is critical. Taking advantage of multiparametic cytometry and unsupervised cell type mapping, here we showed that cells with a myelomonocytic cell surface phenotype—Ly6C⁺, Ly6G⁻—differentiate into five subsets. Similar to previous studies, we confirmed that the recruitment of myelomonocytic cells to the brain is absent in HD, which characterizes a neurodegenerative condition. By contrast, they were present in all different clinical stages of EAE, but their frequency varied. The presence of population D and E with costimulatory molecules and other molecules involved in antigen presentation even in presymptomatic disease, as well as later at the onset and peak of clinical disease, is notable. D and E were not present in the chronic and recovery phase. One implication of these dynamic changes is a role for such cells in initiating adaptive immune responses within the central nervous system.

A determination of the relative influence and functional difference of CNS-resident myeloid cells versus recruited blood-derived myeloid cells in the pathogenesis of different CNS diseases is critical for both understanding pathology and for the development of therapeutic strategies. The role of these recruited cells is poorly understood due to a lack of any specific distinguishing markers.

Previously by preventing the infiltration of blood-derived myeloid cells to the CNS, we proposed that the activation of CNS-resident myeloid cells is required for the initiation of EAE and precedes the entry of blood-derived cells. The progression of EAE (beyond disease onset), however, is due to the entrance of blood-derived myeloid cells. Here, we show that these two cell types have different signaling phenotypes under defined disease conditions. Our data demonstrate signaling differences which distinguish CNS-resident myeloid cells and blood-derived myeloid cells in neuroinflammation. Indeed, the inflammatory attributes of blood-derived myeloid cells were reflected in their cytokine expression profile, where multiple producing cytokine cells—including triple and quadruple cytokines—increased at the onset and peak of the disease in these cells.

These studies illustrate the power of mass cytometry for understanding previously undefined populations of CNS myeloid cells. Their differential behavior in diseases where inflammation is a clear component-EAE, versus a disease where classic inflammation is absent-HD, may allow us to further distinguish between neuroinflammation and neurodegeneration at a molecular level. As we have shown here unexpected therapeutic targets, like α5 integrin are illuminated by this advanced technology for analysis of neuropathology.

### Material and Methods:

Mice. C57BL/6J female mice were purchased from the Jackson Laboratory (Sacramento, Calif.) at 7 weeks. Animals were rested at Stanford University's research animal facility for 2 weeks and were induced EAE at 9 weeks of age. R6/2 female mice were purchased from the Jackson Laboratory at age of 7-8 weeks old and were harvested at 13 weeks of age when they developed severe tremor, irregular gait, abnormal movements and seizures. Animal experiments were approved by, and performed in compliance with, the National Institute of Health guidelines of the Institutional Animal Care and Use Committee at Stanford University. All animals were housed under a 12-hour light cycle. The maximum number of animals housed per cage was five mice. Animals were randomly selected and used in this study.

Induction of EAE in mice by immunization with MOG and adjuvant. EAE was induced in female C57BL/6J mice (the Jackson Laboratory) at 9 weeks of age by subcutaneous immunization in the flank with an emulsion containing 200 μg myelin oligodendrocyte glycoprotein35-55 MOG35-55; MEVGWYRSPFSRVVHLYR NGK) in saline and an equal volume of complete Freund's adjuvant containing 4 μg/ml mycobacterium tuberculosis H37RA (Difco Laboratories Inc., Detroit, Mich.). All mice were administered 400 ng of pertussis toxin (List Biological Laboratories, Inc., Campbell, Calif.) intraperitoneal at 0 and 48 h post-immunization. The neurological impairment was scored as follows: presymptomatic; 10 days post EAE induction with no clinical disease; onset: loss of tail tone and hindlimb weakness, peak; complete hindlimb paralysis, recovered; recovery from hindlimb paralysis and sustaining the improvement, chronic; developed permanent functional impairment after 3-6 month and never recovered.

Antibodies. A summary of antibodies used can be found in tables 1, 2 and 3, including their primary manufacturer, clone, corresponding metal conjugate, and final operating concentration. Antibodies were prepared in amounts varying from 100 to 500 μg at a time using the MaxPAR antibody conjugation kit (Fluidigm, Markham, ON, Canada) following the manufacturer's protocol. After being labeled with their corresponding metal conjugate, the percent yield was determined by measuring their absorbance at 280 nm using a Nanodrop 2000 spectrophotometer (Thermo Scientific, Wilmington, Del.). Antibodies were diluted using Candor PBS Antibody Stabilization solution (Candor Bioscience GmbH, Wangen, Germany) to 0.3 mg/mL, and then stored at 4° C. Each antibody was titrated for optimal staining concentrations using primary murine samples and cell cultures.

Single cell isolation. Mice were deeply anesthetized and monitored. Upon the loss of nociceptive reflexes, animals were perfused transcardially with ice-cold PBS. Brains and spinal cords were removed and gently homogenized in cold HBSS (Life Technologies, 14175-095) on ice. Mononuclear cells were separated with a 30%/70% Percoll (GE Healthcare, Marlborough, Mass.) gradient centrifugation according to previously reported protocol.

Cell suspensions were washed in PBS with 2% FCS and 2 mM EDTA two times and were fixed for 10 min at RT using 1:1.4 proteomic stabilizer according to the manufacturer's instruction (Smart Tube Inc., Palo Alto, Calif.) and frozen at −80° C.

Peripheral blood was collected via the retro-orbital prior to perfusion of the animal and transferred into sodium heparin-coated vacuum tubes 1:1 dilution in RMPI 1640. fixed for 10 min at RT using 1:1.4 proteomic stabilizer according to the manufacturer's instruction (Smart Tube Inc., Palo Alto, Calif.) and frozen at −80° C.

In each experiment, 10-12 mice were pooled in order to provide enough cell number. Each experiment repeated 7 to 10 times from separate immunization and cohort of mice.

Mass-Tag Cell Barcoding. Samples from each condition were Mass-tag Cell Barcoded (MCB). In each sample a unique combination of six palladium isotopes used to encode 20 unique Mass-tag barcodes as previously described61. This technique allows all the samples to be pooled and stained within a single tube, eliminating tube-to-tube variability in antibody staining and minimizing the effect of variable instrument sensitivity. For each sample, 1.5×10⁶ cells from each condition were barcoded. Methanol-permeabilized cells were washed once with Cell Staining Medium (CSM, PBS with 0.5% BSA, 0.02% NaN3) and then once with PBS. Different combinatorial mixtures of Palladium-containing MCB reagents in DMSO were then added to the individual samples at 1:100 DMSO with vortexing and then incubated at room temperature for 15 min, followed by three washes with CSM. The individual samples were then pooled for antibody staining and mass cytometry analysis. After data collection, each condition was deconvoluted using a mass cytometry debarcoding algorithm.

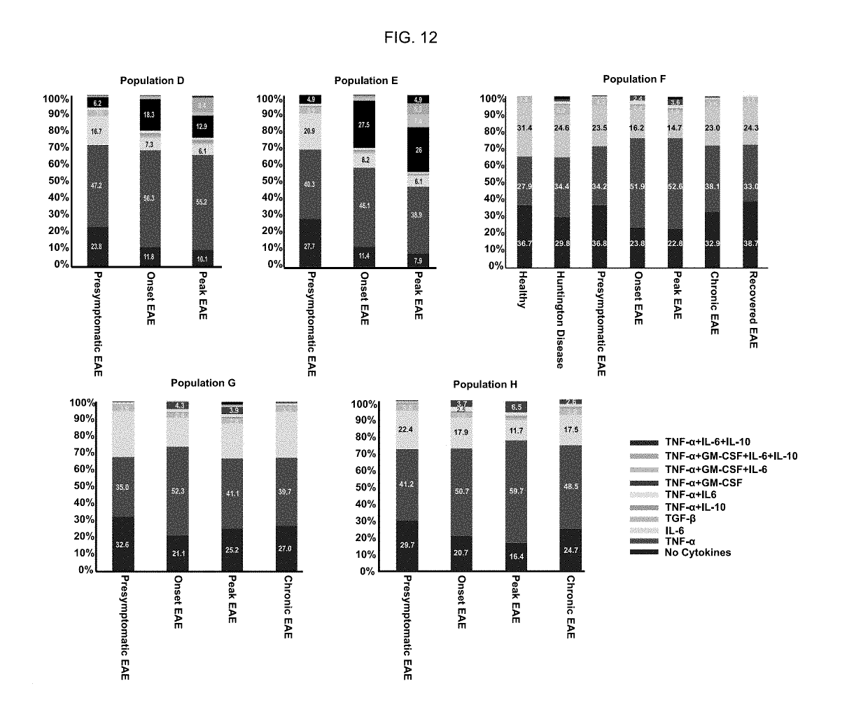

Antibody Staining. Barcoded cells then were resuspended in PBS with 0.5% BSA and 0.02% NaN3 and antibodies against CD16/32 were added at 20 μg/ml for 10 min at RT on a shaker to block Fc receptors. Cells were stained with a cocktail of metal-conjugated surface marker antibodies (FIG. 12), yielding 500 uL final reaction volumes and stained at room temperature for 30 min at RT on a shaker. Following staining, cells were washed 2 times with PBS with 0.5% BSA and 0.02% NaN3. Next, cells were permeabilized with 4° C. methanol for at 10 min at 4° C. Cells were then washed twice in PBS with 0.5% BSA and 0.02% NaN3 to remove remaining methanol. Cells were then stained with intracellular antibodies (Table 1 for signaling experiments and Table 2 for cytokine experiments) in 500 μL for 30 min at RT on a shaker. Sample were then washed twice in PBS with 0.5% BSA and 0.02% NaN3. Cells were incubated overnight at 4° C. with 1 mL of 1:4000 191/193lr DNA intercalator (DVS Sciences/Fluidigm, Markham, ON) diluted in PBS with 1.6% PFA overnight. Following day, cells were washed once with PBS with 0.5% BSA and 0.02% NaN3 and then two times with double-deionized (dd)H2O.

Mass Cytometry Measurement. Prior to analysis, the stained and intercalated cell pellet was resuspended in ddH2O containing polystyrene normalization beads containing lanthanum-139, praseodymium-141, terbium-159, thulium-169 and lutetium-175 as described previously62. Stained cells were analyzed on a CyTOF 2 (Fluidigm, Markham, ON) outfitted with a Super Sampler sample introduction system (Victorian Airship & Scientific Apparatus, Alamo, Calif.)”) at an event rate of 200 to 300 cells per second. All mass cytometry files were normalized together using the mass cytometry data normalization algorithm freely available for download.

Analysis. Clustering: The raw CyTOF data was subject to arsinh(x/5) transformation. We selected cells from each sample which were then pooled together for clustering, generating a dataset with a total of 1,800,183 cells for the signaling dataset and 1,967,893 cells for the cytokine dataset. These datasets were clustered with a novel density-based clustering method known as X-shift. X-shift was developed to compute large multidimensional datasets and automatically determine the optimal number of clusters. In short, X-shift uses the weighted K-nearest neighbor density estimation to find the local maxima of data-point (cell event) density in the multidimensional marker space. X-shift computes the density estimate for each data point and then searches for the local density maxima in a nearest-neighbor graph, which become cluster centroids. All the remaining data points are then connected to the centroids via density-ascending paths in the graph, thus forming clusters. Finally, the algorithm checks for the presence of density minima on a straight line segment between the neighboring centroids, merging closely aligned clusters as necessary. In summary, cells were assigned to different populations based on local gradient of cell event density in the marker expression space. Two cell population counted as separate if cell density in any point on a straight line between centers of populations was lower than density in the population centers. In other words, the peaks of cell event density that represent two populations must be separated by a cleft. Furthermore, clusters separated by a Mahalonobis distance less than 2.0 were merged together. The optimal nearest neighbor parameter, K, was chosen to be 70 in a data-driven manner, by finding the elbow-point of the plot of the number of clusters over K. All data processing was performed with the VorteX clustering environment.

Divisive Marker Tree (DMT) for gating: In order to facilitate back-gating of X-shift clustered populations, we organized the clusters into a Divisive Marker Tree (DMT). The DMT algorithm constructs a binary decision tree that starts with a root node encompassing all clusters; this set of clusters is then subject to iterative binary division. This process results in a hierarchical binary classification of cell types that resembles manual gating hierarchies. By tracing the sequence of marker divisions from the root, we were able to infer a concise marker-based signature for each cell population that differentiates it from other populations.

CD49e (α5 integrin) treatment. EAE mice (n=5 per group) were treated daily with 200 μg of CD49e (α5 integrin) antibody (Clone=5H10-27(MFR5)), or the isotype control (low endotoxin, azide-Free antibody and the isotype control were custom-made by Biolegend for this experiment.) EAE scores were assessed daily for clinical signs of EAE in a blinded fashion without knowing which mouse was receiving treatments. Mice were assessed daily and scored according to: 0, no clinical disease; 1, tail weakness; 2, hindlimb weakness; 3, complete hindlimb paralysis; 4, hindlimb paralysis and some forelimb weakness; 5, moribund or dead. The experiment was concluded due to high morbidity of control mice.

TABLE 1

| | | | Metal | Conc. | Catalog |
|---|---|---|---|---|---|
| Protein | Clone | Manufacturer | Isotope | (μg/mL) | number |
| B220 | RA3-6B2 | BioLegend | Pr141 | 2 | 103202 |
| CD11b | M1/70 | BioLegend | Nd142 | 0.5 | 101202 |
| CD11c | N418 | BioLegend | Nd143 | 8 | 117302 |
| CD194 | 2G12 | BioLegend | Gd160 | 8 | 131202 |
| CD195 | HM-CCR5(7A4) | eBioscience | Gd155 | 8 | 14-1951-85 |
| CD200R | OX2R | BioLegend | Yb172 | 8 | 123902 |
| CD206 | MR5D3 | AbD Serotec | Er166 | 8 | MCA2235 |
| CD217 (IL-17RA) | PAJ-17R | eBioscience | Lu175 | 4 | 12-7182-82 |
| CD274 | B7-H1 | BioLegend | Nd146 | 2 | 124302 |
| CD3 | 145-2C11 | BioLegend | In133 | 4 | 100302 |
| CD38 | 90 | BioLegend | Dy161 | 4 | 102702 |
| CD39 | Duha59 | BioLegend | Er170 | 3 | 143802 |
| CD4 | RM4-5 | BioLegend | Nd150 | 1 | 100506 |
| CD45 | 30-F11 | BioLegend | Yb176 | 1 | 103102 |
| CD49d | 9C10 (MFR4.B) | BioLegend | Sm147 | 4 | 103708 |
| CD49e | 5H10-27 (MFR5) | BioLegend | Nd148 | 4 | 103801 |
| CD80 | 16-101 | BD Pharmigen | Er168 | 4 | 553766 |
| CD86 | GL-1 | BioLegend | Tb159 | 4 | 105002 |
| H-2 | M1/42 | BioLegend | Nd145 | 1 | 125502 |
| Ly6C | HK1.4 | Novus | Eu151 | 1 | NBP1-28046 |
| | | Biologicals | | | |
| Ly6G | 1A8 | BioLegend | ce140 | 2 | 127632 |
| MHCII | M5/114.15.2 | BioLegend | In115 | 2 | 107602 |
| CD317 (PDCA-1) | 120GB | Novous/imgenx | Eu153 | 4 | DDX0390-067 |
| TIM4 | Kat5-18 | Hycult Biotech | Dy163 | 8 | 11550M0512 |
| MerTX | Polyclonal | R&D | Dy162 | 4 | DGS02213111 |
| ALX | Polyclonal | R&D | Er167 | 4 | CTC0213041 |
| TREM2 | 78-18 | BioRad | Tm169 | 4 | 1113 |
| 4D4 | | Collaborator gift* | Sm154 | 0.5 | Gift |
| Fcrls | | Collaborator gift* | Sm152 | 2 | Gift |

TABLE 2

| | | Manu- | Metal | Conc. | Catalog |
|---|---|---|---|---|---|
| Protein | Clone | facturer | Isotope | (μg/mL) | number |
| C/EBPα | D56F10 | CST | Ho165 | 4 | 8178S |
| C/EBPβ | E299 | Abcam | Dy164 | 4 | ab3238 |
| pCREB | 87G3 | CST | Yb174 | 1 | 9198BF |
| pSTAT1 | 58D6 | CST | Gd155 | 4 | 9167BF |
| pSTAT3 | 4/P- | DVS | Gd158 | 1 | 3158005A |
| | STAT3 | | | | |
| pSTAT5 | 47 | BD | Nd144 | 1 | 624084 |
| | | Pharmingen | | | |
| NF-κb (p65) | K10- | BD | Yb171 | 4 | 558393 |
| | 895.12.50 | Pharmingen | | | |
| MAPKAPK2 | 27B7 | CST | Yb173 | 1 | 3007BF |
| cPARP | F21-852 | BD | La139 | 1 | 519000017 |
| | | Pharmingen | | | |

TABLE 3

| | | | Metal | Conc. | Catalog |
|---|---|---|---|---|---|
| Protein | Clone | Manufacturer | Isotope | (μg/mL) | number |
| GM-CSF | MP1-22E9 | BioLegend | Dy164 | 4 | 505402 |
| IFN-a | F1 | Hycult | Yb173 | 4 | HM1001 |
| | | Biotech | | | |
| IFN-g | XMG1.2 | DVS | Ho165 | 4 | 3165003B |
| IL-10 | JES5-16E3 | DVS | Gd158 | 4 | 3158002B |
| IL-17A | TC11- | DVS | Tm169 | 4 | 3169005B |
| | 18H10.1 | | | | |
| IL-6 | MP5-20F3 | DVS | Er167 | 4 | 3167003B |
| TGF- | 19D8 | BioLegend | Yb171 | 4 | 521704 |
| beta | | | | | |
| TNF-a | MP6-XT22 | DVS | Dy162 | 2 | 3162002B |

### Example 2

### Overview of Myeloid Cell Populations

The phenotype of the myeloid cell populations discussed herein are summarized in Table 4. Populations A, B and C correspond to microglial cells. These populations are equivalent to CD45 intermediate, CD11b+ cells in human brains.

In EAE and MS disease and many inflammatory conditions, there is an infiltration of monocytes from peripheral blood. We have identified five monocyte populations in the central nervous system of EAE mice, referred to herein as D, E, F, H, G. In human, these populations correspond to CD11b+CD14+CD16+ monocytes. Cytokine expression profile in these populations shows that in onset of peak of the EAE disease, a percentage of these cells express multiple inflammatory cytokines (TNF-α+GMCSF) compared to healthy state when cells express only one cytokine.

TABLE 4

| Population | CD45 | CD11b | Ly6G | CD49d | CD317 | CD39 | CD86 | MHC II | CD274 | LY6C | CD88 | CD217 |
|---|---|---|---|---|---|---|---|---|---|---|---|---|
| A | intermediate | positive | negative | negative | positive | positive | negative | | | | | |
| B | intermediate | positive | negative | negative | positive | positive | positive | negative | | | | |
| C | intermediate | positive | negative | negative | positive | positive | positive | positive | | | | |
| D | high | positive | negative | positive | | | | positive | positive | positive | | |
| E | high | positive | negative | positive | | | | negative | positive | positive | | |
| F | high | positive | negative | positive | | | | | negative | positive | negative | negative |
| G | high | positive | negative | positive | | | | | negative | positive | positive | negative |
| H | high | positive | negative | positive | | | | | negative | positive | positive | positive |

### Example 3

### Amyotrophic Lateral Sclerosis

Our previous study and others have demonstrated that microglia are the only myeloid cells in brain and spinal cord of mSOD1 mice, a murine model of ALS disease and there is no infiltration of myeloid cells from the peripheral blood (Ajami et al (2007) Nature Neuroscience 10:1538-1543; Chiu et al. (2013) Cell Reports 4(2):385-401). Furthermore, several studies have demonstrated that microglia are involved in the pathogenesis of ALS and restricting the expression of mutant SOD in microglia will delay degeneration and extend survival of motor mSOD-expressing motor neurons (Clement et al (2003) Science 302:113-117; Lino et al (2002) The Journal of Neuroscience 22(12):4825-4832.

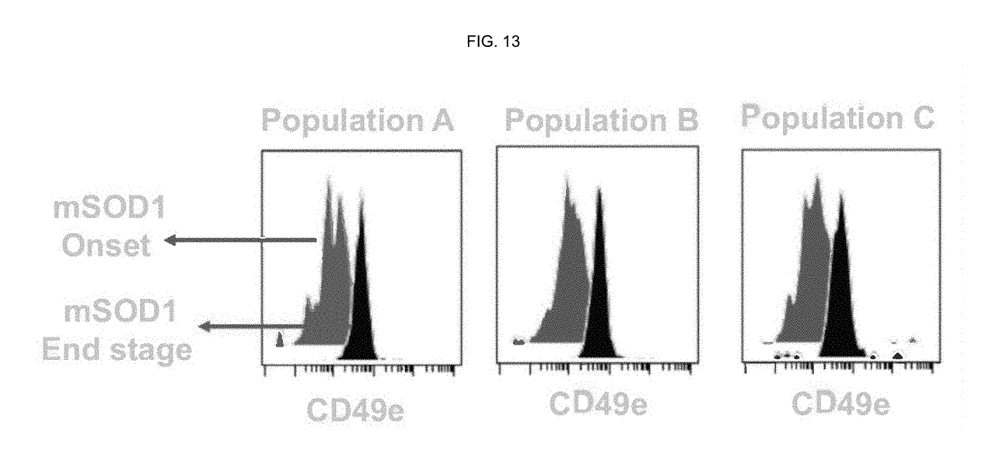

As shown in FIG. 13, there is an increase in CD49e expression in microglia populations at disease end-stage in mice over-expressing human mutant superoxide dismutase 1 (mSOD), a murine model of ALS. We compared the expression level of CD49e (α5 integrin) at disease onset (95 days, start of weight loss based on Boillee et al 2006) to the disease end-stage (140 days, when the mice were completely paralyzed and the experiment had to be terminated). The expression level of CD49e is increased at the disease end stage compare to the onset of the disease.

We compared the frequency of these populations at disease onset (95 days old mice when the weight loss start) and at the disease end-stage (140 days, when mice are completely paralyzed). In disease onset, Population A comprised 2%, population B 5% and population C 2% of the total cell population in CNS. In disease-end stage Population A comprised 4%, population B 12% and population C 2% of the total cell population in CNS. This indicated that population B is increased significantly at the end stage of the disease.

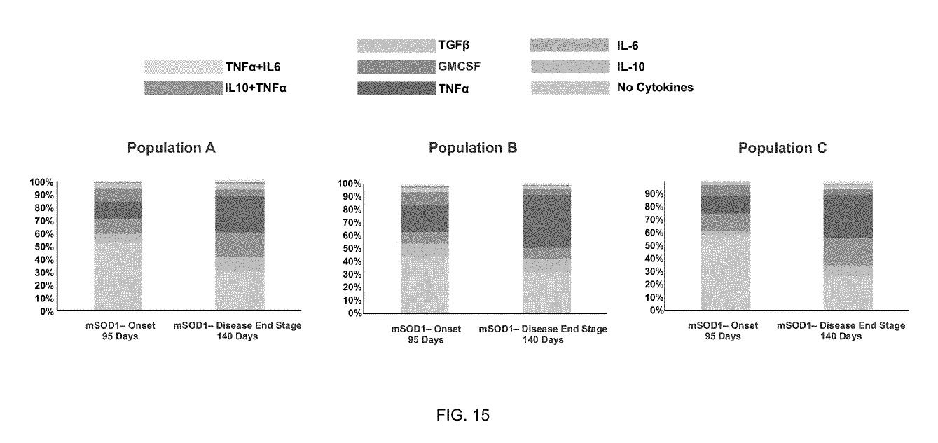

Comparing the cytokine profile of population A, B and C in disease onset and end-stage of disease in mSOD1 mice, demonstrated that population A, B, C express IL-10, IL-6. TNF-α, GMCSF and TGF-beta. Importantly, frequency of the cells expressing TNFα, a major inflammatory cytokine, is increased in disease end-stage in mSOD1 mice. As shown in FIG. 15, in population A, the frequency of TNF-α expressing cells increased from 10% in onset to 30% in end-stage, in population B, the frequency of TNF-α expressing cells increased from 20% in onset of the disease to 40% in end-stage, in population C, the frequency of TNF-α expressing cells increased from 10% to 40%.

Based on this data and previous studies that have demonstrated that microglia are important in disease progression in mSOD1 model of ALS, inhibition of CD49e is a therapeutic target for ALS disease.

To assess treatment, 6-week old mSOD1 mice are treated prior to =disease onset with 100 micrograms anti-CD49e antibody three times per week. The control group is treated with the similar dose of isotype control.

For humans, anti-CD49e is utilized as a treatment for improving motor activity in amyotrophic lateral sclerosis.

### Example 4

### Tattoo Removal

Enhancement of tattoo removal is accomplished by 3× weekly administration systemically, IM, IP intra-dermally, or IV of 100 micrograms of anti-CD49e, for 6 weeks. The regimen may be continued for multiple rounds of therapy beginning one week after each 6 week round.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

What is claimed is:

1. A method for treating an inflammatory disease or condition in a patient, the method comprising:
administering to said patient a therapeutically effective dose of an anti-integrin-α₅ agent.

2. The method of claim 1, wherein the patient is a human.

3. The method of claim 1, wherein the inflammatory disease is multiple sclerosis.

4. A method for treating amyotrophic lateral sclerosis in a patient, the method comprising:
administering to said patient a therapeutically effective dose of an anti-integrin-α₅ agent.

5. The method of claim 1, wherein the anti-integrin-α₅ agent reduces macrophage activity to enhance removal of a tattoo.

6. The method of claim 1, wherein the anti-α₅ agent blocks the binding of integrin α₅ to fibronectin.

7. The method of claim 6, wherein the anti-α₅ agent is an antibody that specifically binds to integrin α₅, integrin β₁, or the heterodimer integrin α₅β₁.

8. The method of claim 7, wherein the antibody is a chimeric or humanized antibody specific for integrin α₅, or a specific binding fragment thereof.

9. The method of claim 8, wherein the antibody comprises a human IgG₄ Fc region.

10. The method of claim 3, further comprising administering an additional therapeutic agent for treatment of multiple sclerosis.

11. The method of claim 10, wherein the additional therapeutic agent is selected from a statin, a cytokine; fingolimod; and copaxone.

12. The method of claim 11, wherein the cytokine is IFNβ.

13. The method of claim 1, wherein the patient is patient is analyzed for responsiveness to cytokine therapy, and where the selection of therapeutic agent is based on such analysis.

14. A composition comprising a package comprising an anti-α₅ agent and a package insert or label that indicates that the anti-α₅ agent is to be administered to a patient for the treatment of a neuroinflammatory disease or ALS.
